SEQUENCE LISTING (27 sequences)

SEQ ID NO: 1 - protein, 9 aa, synthetic construct
GYSITSGYY

SEQ ID NO: 2 - protein, 7 aa, synthetic construct
ITFDGAN

SEQ ID NO: 3 - protein, 12 aa, synthetic construct
TRSSYDYDVLDY

SEQ ID NO: 4 - protein, 6 aa, synthetic construct
QDISNF

SEQ ID NO: 5 - skipped

SEQ ID NO: 6 - protein, 9 aa, synthetic construct
QQGHTLPYT

SEQ ID NO: 7 - protein, 6 aa, synthetic construct
SGYYWN

SEQ ID NO: 8 - protein, 16 aa, synthetic construct
YITFDGANNYNPSLKN

SEQ ID NO: 9 - protein, 10 aa, synthetic construct
SSYDYDVLDY

SEQ ID NO: 10 - protein, 11 aa, synthetic construct
RASQDISNFLN

SEQ ID NO: 11 - protein, 7 aa, synthetic construct
YTSRLHS

SEQ ID NO: 12 - protein, 8 aa, synthetic construct
GYSITSGY

SEQ ID NO: 13 - skipped

SEQ ID NO: 14 - protein, 8 aa, synthetic construct
SYDYDVLD

SEQ ID NO: 15 - protein, 7 aa, synthetic construct
SQDISNF

SEQ ID NO: 16 - protein, 6 aa, synthetic construct
GHTLPY

SEQ ID NO: 17 - protein, 119 aa, synthetic construct
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSS

SEQ ID NO: 18 - protein, 107 aa, synthetic construct
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIK

SEQ ID NO: 19 - protein, 227 aa, synthetic construct
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 20 - protein, 214 aa, synthetic construct
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 22 bp, synthetic construct
  source 1..22: mol_type = other DNA
caatgccatcctggagttcctg

SEQ ID NO: 22 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 23 - DNA, 13993 bp, Homo sapiens
  source 1..13993: mol_type = genomic DNA
tcctggcatcagttactgtgttgactcactcagtgttgggatcactcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggttttttttatcgctgccttgatatacacttttcaaaatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcaggtcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagcgagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtcttttccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctagaatggatttttcccggagccggaagccaggaggaaactacaccacactaaaacattgtctacagctccagatgtttctcattttaaacaactttccactgacaacgaaagtaaagtaaagtattggatttttttaaagggaacatgtgaatgaatacacaggacttattatatcagagtgagtaatcggttggttggttgattgattgattgattgatacattcagcttcctgctgctagcaatgccacgatttagatttaatgatgcttcagtggaaatcaatcagaaggtattctgaccttgtgaacatcagaaggtattttttaactcccaagcagtagcaggacgatgatagggctggagggctatggattcccagcccatccctgtgaaggagtaggccactctttaagtgaaggattggatgattgttcataatacataaagttctctgtaattacaactaaattattatgccctcttctcacagtcaaaaggaactgggtggtttggtttttgttgcttttttagatttattgtcccatgtgggatgagtttttaaatgccacaagacataatttaaaataaataaactttgggaaaaggtgtaaaacagtagccccatcacatttgtgatactgacaggtatcaacccagaagcccatgaactgtgtttccatcctttgcatttctctgcgagtagttccacacaggtttgtaagtaagtaagaaagaaggcaaattgattcaaatgttacaaaaaaacccttcttggtggattagacaggttaaatatataaacaaacaaacaaaaattgctcaaaaaagaggagaaaagctcaagaggaaaagctaaggactggtaggaaaaagctttactctttcatgccattttatttctttttgatttttaaatcattcattcaatagataccaccgtgtgacctataattttgcaaatctgttacctctgacatcaagtgtaattagcttttggagagtgggctgacatcaagtgtaattagcttttggagagtgggttttgtccattattaataattaattaattaacatcaaacacggcttctcatgctatttctacctcactttggttttggggtgttcctgataattgtgcacacctgagttcacagcttcaccacttgtccattgcgttattttctttttcctttataattctttctttttccttcataattttcaaaagaaaacccaaagctctaaggtaacaaattaccaaattacatgaagatttggtttttgtcttgcatttttttcctttatgtgacgctggaccttttctttacccaaggatttttaaaactcagatttaaaacaaggggttactttacatcctactaagaagtttaagtaagtaagtttcattctaaaatcagaggtaaatagagtgcataaataattttgttttaatctttttgtttttcttttagacacattagctctggagtgagtctgtcataatatttgaacaaaaattgagagctttattgctgcattttaagcataattaatttggacattatttcgtgttgtgttctttataaccaccaagtattaaactgtaaatcataatgtaactgaagcataaacatcacatggcatgttttgtcattgttttcaggtactgagttcttacttgagtatcataatatattgtgttttaacaccaacactgtaacatttacgaattatttttttaaacttcagttttactgcattttcacaacatatcagacttcaccaaatatatgccttactattgtattatagtactgctttactgtgtatctcaataaagcacgcagttatgttac

SEQ ID NO: 24 - DNA, 176 bp, Homo sapiens
  source 1..176: mol_type = genomic DNA
gaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagag

SEQ ID NO: 25 - DNA, 22 bp, synthetic construct
  source 1..22: mol_type = other DNA
caggaactccaggatggcattg

SEQ ID NO: 26 - DNA, 236 bp, Homo sapiens
  source 1..236: mol_type = genomic DNA
tttttgttttgcctttttggtatcttacaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggtagggcgacagatctaataggaatgaaaa

SEQ ID NO: 27 - protein, 9 aa, synthetic construct
DYEKHKVYA

(19) United States
(12) Patent Application Publication
(10) Pub. No.: US 2026/0183410 A1
(43) Pub. Date: Jul. 2, 2026

(54) MUSCLE TARGETING COMPLEXES AND USES THEREOF FOR SKIPPING EXON 45 OF A DMD GENE

(71) Applicant: Dyne Therapeutics, Inc., Waltham, MA (US)

(72) Inventors: Romesh R. Subramanian, Framingham, MA (US); Mohammed T. Qatanani, Waltham, MA (US); Timothy Weeden, Waltham, MA (US); Cody A. Desjardins, Waltham, MA (US); Brendan Quinn, Waltham, MA (US); John Najim, Waltham, MA (US)

(73) Assignee: Dyne Therapeutics, Inc., Waltham, MA (US)

(21) Appl. No.: 19/125,492

(22) PCT Filed: Oct. 30, 2023
(86) PCT No.: PCT/US2023/078169

§ 371 Date: Apr. 29, 2025

Related U.S. Application Data
(60) Provisional application No. 63/381,730, filed on Oct. 31, 2022.

(51) Int. Cl. A61K 47/68; A61P 21/00; C07K 16/28; C12N 15/113

(52) U.S. Cl. CPC A61K 47/6807; A61K 47/6849; A61K 47/6889; A61P 21/00; C07K 16/2881; C12N 15/113; C12N 2310/11; C12N 2310/3233; C12N 2310/3513; C12N 2320/32; C12N 2320/33

(57) ABSTRACT

Aspects of the disclosure relate to complexes and other aspects relate to formulations (e.g., aqueous, lyophilized forms) comprising such complexes (e.g, wherein each complex is of the exemplary formula shown below) comprising a phosphorodiamidate morpholino oligomer (e.g, useful for targeting DMD) covalently linked to an antibody (e.g., anti-TfR1 antibody). Also provided are uses of these formulations for treating a subject having a mutated DMD allele associated with Duchenne Muscular Dystrophy. (I)

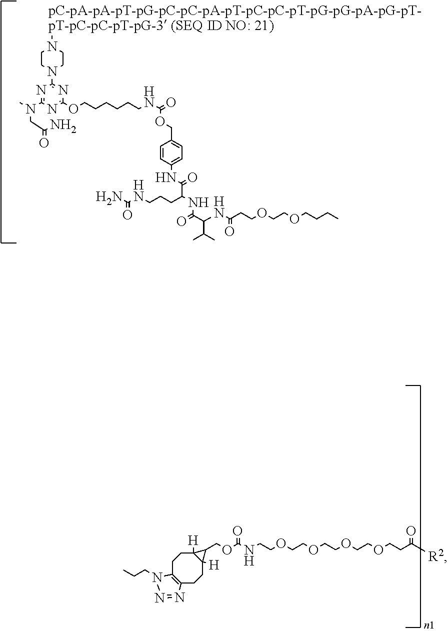

### RELATED APPLICATIONS

This application claims priority under 35 U.S.C. § 119(e) to U.S. Provisional Patent Application No. 63/381,730, filed Oct. 31, 2022, entitled “MUSCLE TARGETING COMPLEXES AND USES THEREOF FOR SKIPPING EXON 45 OF A DMD GENE,” the entire contents of which are herein incorporated by reference.

### FIELD OF THE INVENTION

The present application relates to targeting complexes for delivering molecular payloads (e.g., oligonucleotides) to cells, formulations comprising such complexes, and uses thereof, particularly uses relating to treatment of disease.

### REFERENCE TO AN ELECTRONIC SEQUENCE LISTING

The contents of the electronic sequence listing (D082470083W000-SEQ-COB.xml; Size: 38,671 bytes; and Date of Creation: Oct. 30, 2023) are herein incorporated by reference in their entirety.

### BACKGROUND

Dystrophinopathies are a group of distinct neuromuscular diseases that result from mutations in dystrophin gene. Dystrophinopathies include Duchenne muscular dystrophy, Becker muscular dystrophy, and X-linked dilated cardiomyopathy. DMD, which encodes dystrophin, is a large gene, containing 79 exons and approximately 2.6 million total base pairs. Numerous mutations in DMD, including exonic frameshift, deletion, substitution, and duplicative mutations, are able to diminish the expression of functional dystrophin, leading to dystrophinopathies.

### SUMMARY

According to some aspects, the present disclosure provides muscle targeting complexes useful for promoting expression or activity of a dystrophin protein (e.g., a truncated dystrophin protein) and/or methods of treating Duchenne Muscular Dystrophy in a subject. The truncated dystrophin protein is functional (e.g., retains activities of a wild-type dystrophin protein). In some embodiments, the truncated dystrophin protein retains partial function of a wild-type dystrophin protein.

According to some aspects, complexes comprising a structure of formula (I): [R¹]ₙ₁—R² are provided herein. In some embodiments, each R¹ comprises a group of the formula (Ia):

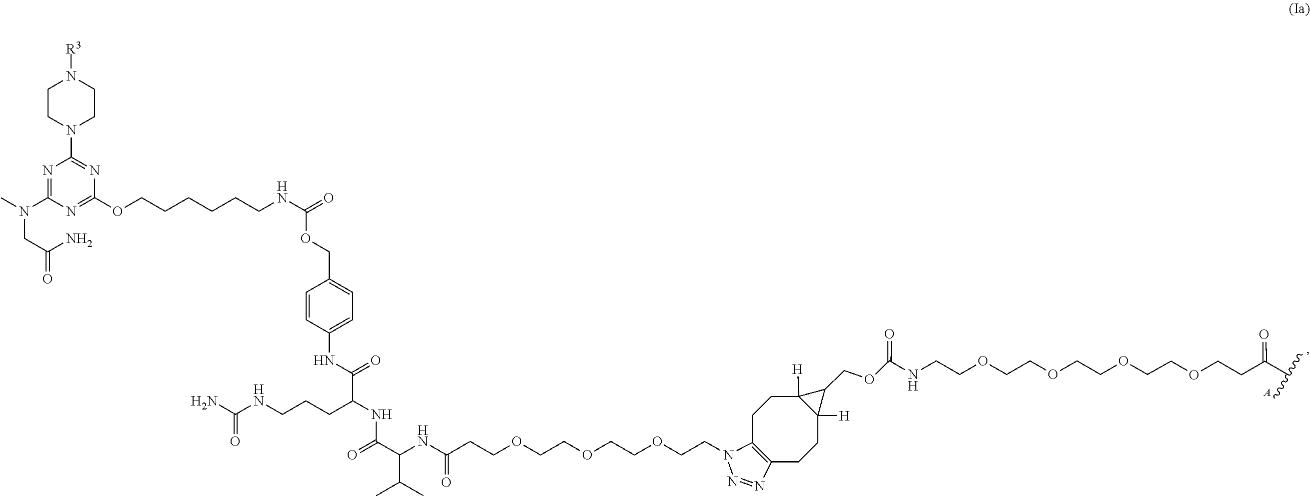

wherein R³ comprises a phosphorodiamidate morpholino oligomer (PMO) comprising the base sequence of CAATGCCATCCTGGAGTTCCTG (SEQ ID NO: 21);
wherein R² comprises an anti-transferrin receptor 1 (anti-TfR1) antibody comprising a heavy chain complementarity determining region 1 (CDR-H1), a heavy chain complementarity determining region 2 (CDR-H2), a heavy chain complementarity determining region 3 (CDR-113), a light chain complementarity determining region 1 (CDR-L1), a light chain complementarity determining region 2 (CDR-L2), and a light chain complementarity determining region 3 (CDR-L3) selected from Table 2,
wherein R¹ is covalently linked to R² at attachment point A; and wherein n1 is an integer of one or greater representing the number of instances of R¹ in the complex, wherein each instance of R¹ is covalently linked to a different amino acid residue of the anti-TfR1 antibody.

In some embodiments, each R¹ comprises a group of the formula (Ib):

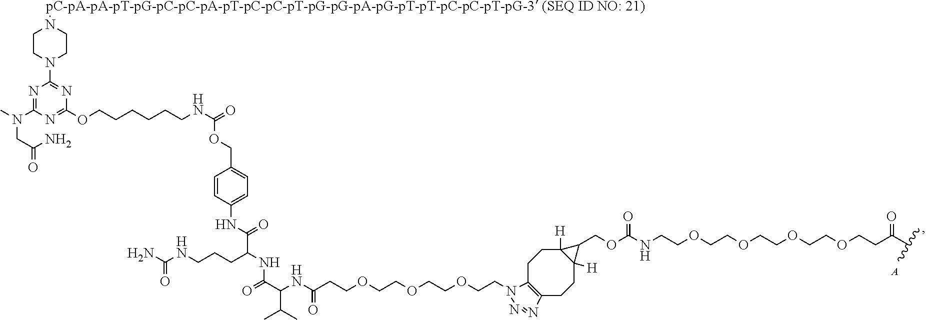

in which -p is a phosphorodiamidate linkage of a phosphorodiamidate morpholino oligomer (PMO), and wherein the PMO comprises a base sequence of

(SEQ ID NO: 21)
CAATGCCATCCTGGAGTTCCTG;

wherein R² comprises an anti-TfR1 antibody comprising a CDR-H1, a CDR-H2, a CDR-H3, a CDR-L1, a CDR-L2, and a CDR-L3 selected from Table 2;
wherein R¹ is covalently linked to R² at attachment point A; and wherein n1 is an integer of one or greater representing the number of instances of R¹, wherein each instance of R¹ is covalently linked to a different amino acid residue of the anti-TfR1 antibody.

In some embodiments, each R¹ comprises a group of the formula (Ic):

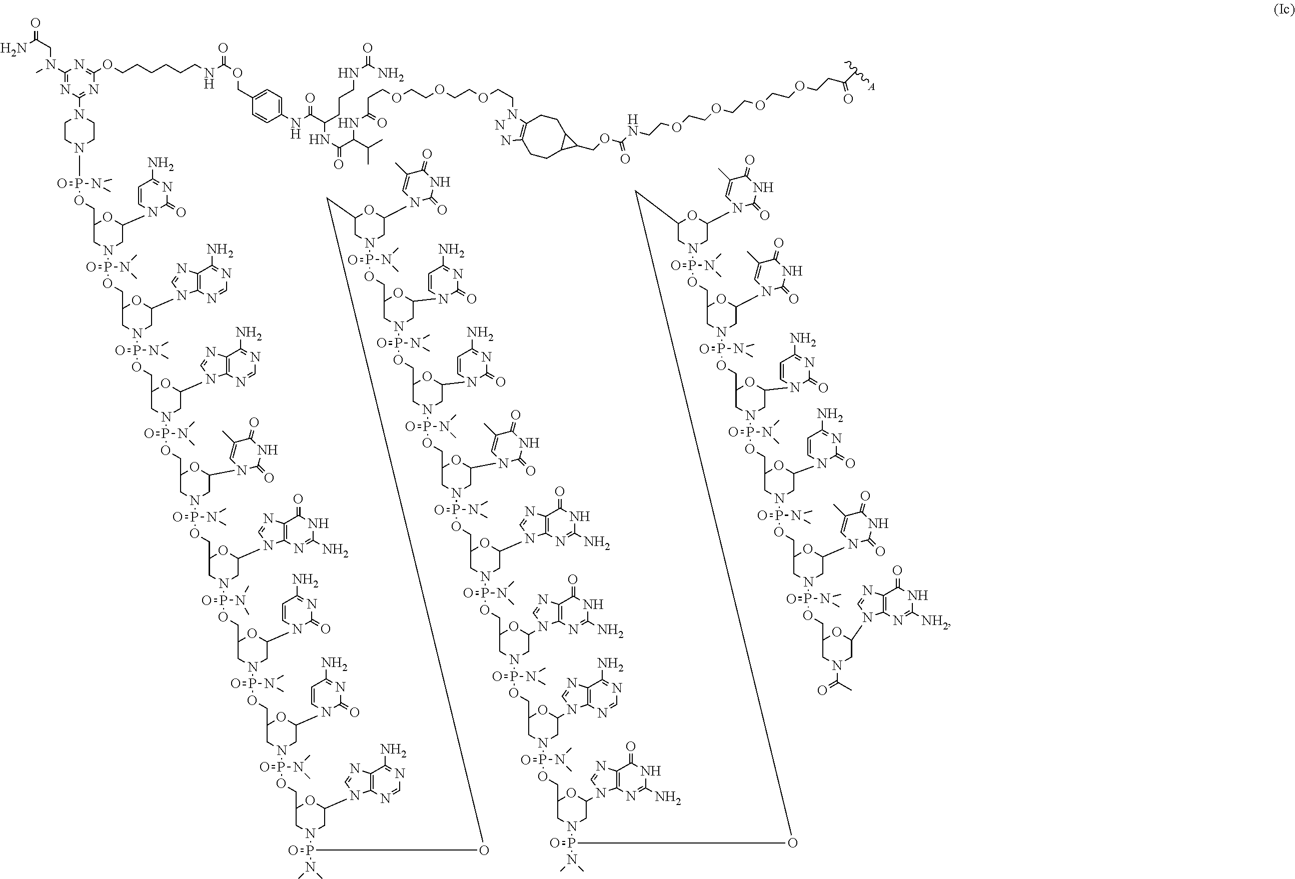

wherein R² comprises an anti-TfR1 antibody comprising a CDR-H1, a CDR-H2, a CDR-H3, a CDR-L1, a CDR-L2, and a CDR-L3 selected from Table 2;
wherein R¹ is covalently linked to R² at attachment point A; and wherein n1 is an integer of one or greater representing the number of instances of R¹, wherein each instance of R¹ is covalently linked to a different amino acid residue of the anti-TfR1 antibody.

In some embodiments, a complex comprises a structure of formula (Id):

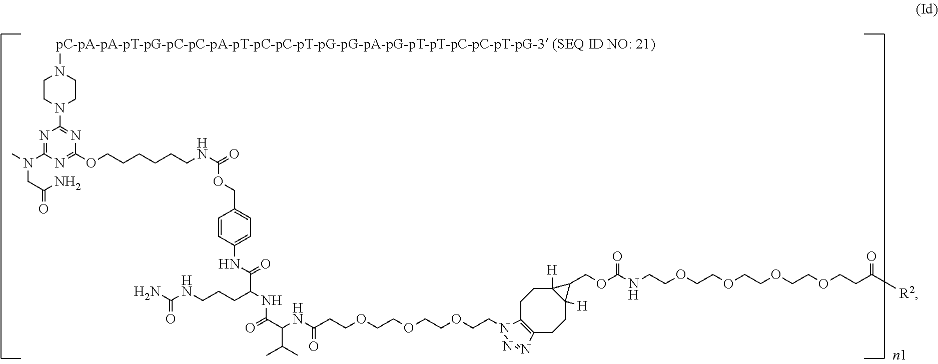

in which -p is a phosphorodiamidate linkage of a phosphorodiamidate morpholino oligomer (PMO), and wherein the PMO comprises a base sequence of

(SEQ ID NO: 21)
CAATGCCATCCTGGAGTTCCTG;

wherein R² comprises an anti-TfR1 antibody comprising a CDR-H1, a CDR-H2, a CDR-H3, a CDR-L1, a CDR-L2, and a CDR-L3 selected from Table 2,
wherein each instance of the group enclosed by square brackets in formula (Id) is covalently linked to a different amino acid residue of the anti-TfR1 antibody; and wherein n1 is an integer of one or greater representing the number of instances of the group enclosed by square brackets in formula (Id).

In some embodiments, the anti-TfR1 antibody is a Fab fragment, a full-length IgG, a Fab′ fragment, or a F(ab′)₂ fragment. In some embodiments, the anti-TfR1 antibody is a Fab fragment.

In some embodiments, the anti-TfR1 antibody comprises a VH comprising the amino acid sequence of SEQ ID NO: 17 and a VL comprising the amino acid sequence of SEQ ID NO: 18. In some embodiments, the anti-TfR1 antibody comprises a heavy chain comprising the amino acid sequence of SEQ ID NO: 19 and a light chain comprising the amino acid sequence of SEQ ID NO: 20.

In some embodiments, each instance of R¹ is covalently linked to a different lysine residue of the anti-TfR1 antibody.

In some embodiments, the different amino acid residues comprise K188 and K190 of the light chain constant region based on Kabat numbering.

In some embodiments, the different amino acid residues are represented by lysine (K) residues in a sequence motif DYEKHKVYA (SEQ ID NO: 27) of the light chain constant region of the anti-TfR1 antibody.

According to some aspects, compositions comprising a complex disclosed herein are provided. In some embodiments, the composition is in the form of an aqueous solution.

In some embodiments, the anti-TfR1 antibodies of the complexes in the composition comprise light chain constant regions, and at least 80% of the light chain constant regions of the anti-TfR1 antibodies of the complexes in the composition are independently covalently linked to an oligonucleotide at a linkage site represented by K188 (based on Kabat numbering) and/or a linkage site represented by K190 (based on Kabat numbering) of the light chain constant region of each anti-TfR1 antibody.

In some embodiments, the anti-TfR1 antibodies of the complexes in the composition comprise light chain constant regions, and at least 80% of light chain constant regions of the anti-TfR1 antibodies of the complexes in the composition are independently covalently linked to an oligonucleotide at linkage sites represented by lysine (K) residues in a sequence motif DYEKHKVYA (SEQ ID NO: 27) of the light chain constant region of each antibody.

According to some aspects, methods of promoting expression or activity of a dystrophin protein in a subject are provided herein. In some embodiments, the method comprises administering to the subject a complex or composition disclosed herein.

In some embodiments, the dystrophin protein is a truncated dystrophin protein.

According to some aspects, methods of treating a subject having a mutated DMD allele associated with Duchenne Muscular Dystrophy are provided herein. In some embodiments, the method comprises administering to the subject a complex or composition disclosed herein.

In some embodiments, the complex promotes expression or activity of a dystrophin protein in the subject. In some embodiments, the dystrophin protein is a truncated dystrophin protein.

In some embodiments, the mutated DMD allele comprises a mutation amenable to exon 45 skipping.

In some embodiments, the mutated DMD allele comprises a frameshift mutation in exon 45.

### BRIEF DESCRIPTION OF THE DRAWINGS

FIG. 1 shows data illustrating that systemic administration of conjugates containing an anti-TfR1 Fab covalently linked to an exon 45-targeting oligonucleotide (ASO) achieved higher levels of ASO in muscle tissues in non-human primates, when compared to systemic administration of ASO not covalently linked to a Fab.

FIG. 2 shows exon 45 skipping in human myotubes, either wild-type (“WT Myotubes”) or comprising a mutation amenable to exon 45 skipping introduced by CRISPR/Cas genome editing (“Del46 Myotubes”).

FIG. 3 shows tissue exposure (ng/g) in the quadriceps (“Quad”), gastrocnemius (“Gastroc”), heart (“Heart”), and diaphragm (“Dia”) of hTfR1/hDMD_{WT}/mdx mice 7 days following intravenous administration of a DMD-targeting oligonucleotide (ASO) comprised in an anti-TfR1 Fab-ASO conjugate.

FIGS. 4A-4D show exon 45 skipping in muscle tissue of hTfR1/hDMD_{WT}/mdx mice treated intravenously with vehicle control (“Veh.”) or an anti-TfR1 Fab-ASO conjugate (“Anti-TfR1 Fab-ASO Conjugate”) 7 days following treatment. Exon skipping is shown in quadriceps (FIG. 4A), gastrocnemius (FIG. 4B), heart (FIG. 4C), and diaphragm (FIG. 4D).

### DETAILED DESCRIPTION OF CERTAIN EMBODIMENTS

According to some aspects, the present disclosure provides muscle targeting complexes useful for promoting expression or activity of a dystrophin protein (e.g., a truncated dystrophin protein) and/or methods of treating Duchenne Muscular Dystrophy in a subject. The truncated dystrophin protein is functional (e.g., retains activities of a wild-type dystrophin protein). In some embodiments, the truncated dystrophin protein retains partial function of a wild-type dystrophin protein. In some embodiments, the muscle targeting complexes comprise a muscle targeting agent (e.g., an anti-TfR1 antibody) covalently linked to an oligonucleotide (e.g., a PMO). In some embodiments, the oligonucleotide comprises a region of complementarity to a DMD sequence.

According to some aspects, the present disclosure provides compositions comprising a plurality of complexes. In some embodiments, a complex of the compositions described herein comprises an antibody (e.g., an anti-transferrin receptor 1 (TfR1)) covalently linked to one or more oligonucleotides. In some embodiments, the antibody comprises a heavy chain comprising a heavy chain variable region (VH) and a heavy chain constant region, and a light chain comprising a light chain variable region (VL) and a light chain constant region. In some embodiments, each of the one or more oligonucleotides is covalently linked to a different amino acid residue of the antibody, such as a lysine residue. In some embodiments, light chain constant regions of antibodies of complexes in the composition are independently covalently linked to an oligonucleotide at a linkage site represented by K188 (based on Kabat numbering) and/or a linkage site represented by K190 (based on Kabat numbering) of the light chain constant regions of the antibodies. In some embodiments, a light chain of an antibody of a complex is covalently linked to an oligonucleotide at a linkage site represented by a lysine (K) residue in a sequence motif DYEKHKVYA (SEQ ID NO: 27) of the light chain.

### Definitions

Administering: As used herein, the terms “administering” or “administration” means to provide a complex to a subject in a manner that is physiologically and/or (e.g., and) pharmacologically useful (e.g., to treat a condition in the subject).

Approximately: As used herein, the term “approximately” or “about,” as applied to one or more values of interest, refers to a value that is similar to a stated reference value. In certain embodiments, the term “approximately” or “about” refers to a range of values that fall within 15%1, 4%1, 3%1, 12%, 1%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a Fab′ fragment, a F(ab′)₂ fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgAQ1, IgA2, IgD, IgM, and IgE constant domains. In some embodiments, an antibody comprises a heavy (H) chain variable region (abbreviated herein as VH), and/or (e.g., and) a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or (e.g., and) CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or (e.g., and) the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information system® www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33:D593-597 (2005); Lefranc, M.-P. et al., Nucleic Acids Res., 37:D1006-1012 (2009); Lefranc, M.-P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems. Examples of CDR definition systems are provided in Table 1.

TABLE 1
CDR Definitions

| | IMGT¹ | Kabat² | Chothia³ |
|---|---|---|---|
| CDR-H1 | 27-38 | 31-35 | 26-32 |
| CDR-H2 | 56-65 | 50-65 | 53-55 |
| CDR-H3 | 105-116/117 | 95-102 | 96-101 |
| CDR-L1 | 27-38 | 24-34 | 26-32 |
| CDR-L2 | 56-65 | 50-56 | 50-52 |
| CDR-L3 | 105-116/117 | 89-97 | 91-96 |

¹IMGT ^{®}, the international ImMunoGeneTics information system ^{®}, imgt.org, Lefranc, M.-P. et al., Nucleic Acids Res., 27:209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196:901-917 (1987))

Complementary: As used herein, the term “complementary” refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Covalently linked: As used herein, the term “covalently linked” refers to a characteristic of two or more molecules being linked together via at least one covalent bond. In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.

DMD allele: As used herein, the term “DMD allele” refers to any one of alternative forms (e.g., wild-type or mutant forms) of a DMD gene. In some embodiments, a DMD allele may encode for dystrophin that retains its normal and typical functions. In some embodiments, a DMD allele may comprise one or more mutations that results in muscular dystrophy. Common mutations that lead to Duchenne muscular dystrophy involve frameshift, deletion, substitution, and duplicative mutations of one or more of 79 exons present in a dystrophin allele, e.g., exon 8, exon 23, exon 41, exon 44, exon 50, exon 51, exon 52, exon 53, or exon 55. Further examples of DMD mutations are disclosed, for example, in Flanigan K M, et al., Mutational spectrum of DMD mutations in dystrophinopathy patients: application of modern diagnostic techniques to a large cohort. Hum Mutat. 2009 December; 30 (12):1657-66, the contents of which are incorporated herein by reference in its entirety.

Duchenne Muscular Dystrophy: As used herein the term “Duchenne Muscular Dystrophy” refers toa muscle disease that results from a mutation in the DMD allele located on the X chromosome, locus Xp21. Symptoms of Duchenne muscular dystrophy include muscle loss or degeneration, diminished muscle function, pseudohypertrophy of the tongue and calf muscles, higher risk of neurological abnormalities, and a shortened lifespan. Duchenne muscular dystrophy is associated with Online Mendelian Inheritance in Man (OMIM) Entry #310200.

Dystrophin: As used herein the term “dystrophin” refers to any one of alternative forms (e.g., wild-type or mutant forms) of a dystrophin protein. Dystrophin is rod-shaped cytoplasmic protein, that is part of a protein complex connecting the intracellular cytoskeleton of a muscle fiber to the extracellular matrix. The absence of dystrophin results in Duchenne Muscular Dystrophy.

Dystrophinopathy: As used herein, the term “dystrophinopathy” refers to a muscle disease that results from one or more mutated DMD alleles. Dystrophinopathies include a spectrum of conditions (ranging from mild to severe) that includes Duchenne muscular dystrophy, Becker muscular dystrophy, and DMD-associated dilated cardiomyopathy (DCM). In some embodiments, at one end of the spectrum, dystrophinopathy is phenotypically associated with an asymptomatic increase in serum concentration of creatine phosphokinase (CK) and/or (e.g., and) muscle cramps with myoglobinuria. In some embodiments, at the other end of the spectrum, dystrophinopathy is phenotypically associated with progressive muscle diseases that are generally classified as Duchenne or Becker muscular dystrophy when skeletal muscle is primarily affected and as DMD-associated dilated cardiomyopathy (DCM) when the heart is primarily affected. Symptoms of Duchenne muscular dystrophy include muscle loss or degeneration, diminished muscle function, pseudohypertrophy of the tongue and calf muscles, higher risk of neurological abnormalities, and a shortened lifespan. Duchenne muscular dystrophy is associated with Online Mendelian Inheritance in Man (OMIM) Entry #310200. Becker muscular dystrophy is associated with OMIM Entry #300376. Dilated cardiomyopathy is associated with OMIM Entry X #302045.

Exonic splicing enhancer (ESE): As used herein, the term “exonic splicing enhancer” or “ESE” refers to a nucleic acid sequence motif within an exon of a gene, pre-mRNA, or mRNA that directs or enhances splicing of pre-mRNA into mRNA, e.g., as described in Blencowe et al., Trends Biochem Sci 25, 106-10. (2000), incorporated herein by reference. ESEs are splicing features. ESEs may direct or enhance splicing, for example, to remove one or more introns and/or one or more exons from a gene transcript. ESE motifs are typically 6-8 nucleobases in length. SR proteins (e.g., proteins encoded by the gene SRSF1, SRSF2, SRSF3, SRSF4, SRSF5, SRSF6, SRSF7, SRSF8, SRSF9, SRSF10, SRSF 11, SRSF12, TRA2A or TRA2B) bind to ESEs through their RNA recognition motif region to facilitate splicing. ESE motifs can be identified through a number of methods, including those described in Cartegni et al., Nucleic Acids Research, 2003, Vol. 31, No. 13, 3568-3571, incorporated herein by reference.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or (e.g., and) VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. In one embodiment, humanized anti-transferrin receptor antibodies and antigen binding portions are provided. Such antibodies may be generated by obtaining murine anti-transferrin receptor monoclonal antibodies using traditional hybridoma technology followed by humanization using in vitro genetic engineering, such as those disclosed in Kasaian et al PCT publication No. WO 2005/123126 A2.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NYAcad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Morpholinos: As used herein, the term “morpholino”, also referred to as a “phosphorodiamidate morpholino oligomer”, refers to a molecular structure that contains nucleobases attached to a backbone of methylenemorpholine rings linked through a phosphorodiamidate group. In some embodiments, the oligonucleotide may be a morpholino-based compounds. Morpholino-based oligomeric compounds are described in Dwaine A.

Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidate morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleosides (e.g., 2′-O-methyl sugar modifications, purine or pyrimidine modifications). In some embodiments, an oligonucleotide may comprise one or more modified internucleoside linkage. In some embodiments, an oligonucleotide may comprise one or more phosphorothioate linkages, which may be in the Rp or Sp stereochemical conformation.

Region of complementarity: As used herein, the term “region of complementarity” refers to a nucleotide sequence, e.g., of an oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻¹ M, 10⁻⁹ M, 10⁻¹⁰ M, 10⁻¹¹ M, 10⁻¹² M, 10⁻¹³ M, or less. In some embodiments, an antibody specifically binds to the transferrin receptor, e.g., an epitope of the apical domain of transferrin receptor.

Splice acceptor site: As used herein, the term “splice acceptor site” or “splice acceptor” refers to a nucleic acid sequence motif at the 3′ end of an intron or across an intron/exon junction of a gene or pre-mRNA that is involved in splicing of pre-mRNA into mRNA (i.e., removing introns from the pre-mRNA), and can be referred to as a splicing feature. A splice acceptor site includes a terminal AG sequence at the 3′ end of an intron, which is typically preceded (5′-ward) by a region high in pyrimidines (C/U). Upstream from the splice acceptor site is the branch point. Formation of a lariat loop intermediate structure by a transesterification reaction between the branch point and the splice donor site releases a 3′-OH of the 5′ exon, which subsequently reacts with the first nucleotide of the 3′ exon, thereby joining the exons and releasing the intron lariat. The AG sequence at the 3′ end of the intron in the splice acceptor site is known to be critical for proper splicing, as changing one of these nucleotides results in inhibition of splicing. Rarely, alternative splice acceptor sites have an AC at the 3′ end of the intron, instead of the more common AG. A common splice acceptor site motif has a sequence of or similar to [Y-rich region]-NCAGG or YxNYAGG, in which Y represents a pyrimidine, N represents any nucleotide, and x is a number from 4 to 20. The cut site follows the AG, which represent the 3′-terminal nucleotides of the excised intron.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having a disease resulting from a mutated DMD gene sequence, e.g., a mutation in an exon of a DMD gene sequence. In some embodiments, a subject has a dystrophinopathy, e.g., Duchenne muscular dystrophy.

Transferrin receptor: As used herein, the term, “transferrin receptor” (also known as TFRC, CD71, p90, TFR or TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).

Ranges: All ranges provided in the present disclosure are inclusive of the end points.

### Complexes

Provided herein are complexes comprising an antibody covalently linked to an oligonucleotide. In some embodiments, a complex comprises a muscle-targeting antibody (e.g., an anti-TfR1 antibody) covalently linked to one or more oligonucleotides. In some embodiments, the oligonucleotide is a PMO. In some embodiments, the oligonucleotide is an oligonucleotide that targets a mutated DMD allele to promote exon skipping (e.g., to promote skipping of exon 45). Complexes disclosed herein are useful in methods of promoting expression or activity of a dystrophin protein and/or treating Duchenne Muscular Dystrophy in a subject comprising administering to the subject an effective amount of the complexes.

Complexes disclosed herein generally comprise a linker that covalently links an antibody disclosed herein (e.g., an anti-TfR1 antibody disclosed herein) to an oligonucleotide (e.g., a PMO). A linker comprises at least one covalent bond.

In some embodiments, a light chain constant region of an antibody of a complex is covalently linked to an oligonucleotide at a linkage site represented by K188 (based on Kabat numbering) and/or a linkage site represented by K190 (based on Kabat numbering) of the light chain constant region. In some embodiments, a light chain of an antibody of a complex is covalently linked to an oligonucleotide at a linkage site represented by a lysine (K) residue in a sequence motif DYEKHKVYA (SEQ ID NO: 27) of the light chain.

In some embodiments, complexes disclosed herein comprise a structure of formula (I): [R¹]ₙ₁—R², in which each R¹ independently comprises a compound comprising an oligonucleotide (e.g., a PMO) and R² comprises an anti-TfR1 antibody, and wherein in each complex n1 is independently an integer (e.g., of one or greater) representing the number of instances of R¹ in each complex. In some embodiments, each R¹ independently comprises a group comprising an oligonucleotide. In some embodiments, each R¹ independently comprises a group that comprises additional elements in addition to an oligonucleotide. In some embodiments, R² comprises an anti-TfR1 antibody. In some embodiments, R² is comprises anti-TfR1 Fab.

In some embodiments in which R² comprises an anti-TfR1 antibody, the antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the antibody comprises a VH comprising the amino acid sequence of SEQ ID NO: 17 and/or comprises a VL comprising the amino acid sequence of SEQ ID NO: 18. In some embodiments, the antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)₂ fragment, an scFv, or an Fv. In some embodiments, the antibody is a Fab fragment.

In some embodiments, the value of n1 of each or any complex (e.g., any complex in any of the compositions or methods disclosed herein) is an integer from one up to the number of amino acid residues in the antibody to which conjugation is desired or targeted (e.g., the number of lysine residues in the antibody). In some embodiments, in each complex the value of n1 is independently selected from 1, 2, 3, 4, 5, 6, 7, 8, 9, 10, 11, 12, 13, 14, 15, 16, 17, 18, 19, 20, 21, 22, 23, 24, 25, 26, and 27. In some embodiments, in each complex the value of n1 is independently selected from 1, 2, 3, 4, 5, 6, 7, 8, 9, 10, 11, 12, 13, 14, 15, 16, 17, 18, 19, 20, 21, 22, 23, 24, 25 and 26. In some embodiments, in each complex the value of n1 is independently in the range of 1-27, 1-26, 1-10, 1-5, or 1-3.

In some embodiments, complexes described herein are presented as compositions (e.g., in aqueous solutions) for administration to a subject. In some embodiments, the composition comprises a plurality of complexes. In some embodiments, the plurality of complexes each comprise a common targeting agent (e.g. an antibody) and a common oligonucleotide (e.g., a PMO). In such embodiments, different complex types are characterized by having different numbers of oligonucleotides covalently linked to an antibody. For example, in some embodiments, a composition for administration to a subject comprises a plurality of complex types in which each complex type comprises a structure of formula (I): [R¹]ₙ₁—R², in which each R¹ independently comprises a compound comprising an oligonucleotide (e.g., a PMO) and R² comprises an anti-TfR1 antibody, and in which in each complex type n1 is independently an integer of one or greater representing the number of instances of R¹ in each complex of the complex type, and in which the different complex types of the composition are characterized by having different n1 values (e.g., n1 values in the range of 1-27, 1-26, 1-25, 1-20, 1-15, 1-10, 1-5, or 1-3).

In some embodiments, light chain constant regions of antibodies of complexes in a composition are independently covalently linked to an oligonucleotide at a linkage site represented by K188 (based on Kabat numbering) and/or a linkage site represented by K190 (based on Kabat numbering) of the light chain constant regions of the antibodies. In some embodiments, light chains of antibodies of complexes in a composition are independently covalently linked to an oligonucleotide at linkage sites represented by lysine (K) residues in a sequence motif DYEKHKVYA (SEQ ID NO: 27) of the light chains of the antibodies.

In some embodiments, a composition for administration to a subject in the methods described herein comprises unconjugated antibody (e.g., in trace amounts) and antibody conjugated to one or more oligonucleotides. In some embodiments, unconjugated antibody may be referred to as a compound of a structure of formula (I): [R¹]ₙ₁—R², for which n1 is zero. Accordingly, in some embodiments, a composition for administration to a subject in the methods described herein comprises compounds (e.g., complexes) of the structure of formula (I): [R¹]ₙ₁—R², for which each R¹ independently comprises a group comprising an oligonucleotide, R² comprises an anti-TfR1 antibody and n1 is independently an integer of zero or greater that reflects the number of instances of R¹ in each compound (e.g., complex). In some embodiments, the fraction of compounds of the structure formula (I): [R¹]ₙ₁—R², in a composition, for which n1 is zero, compared with all compounds of that structure in the composition for which n1 is one or greater, is less than 10%, less than 5%, less than 1% less than 0.5%, less than 0.1%, less than 0.05%, or less than 0.01%.

In some embodiments, each instance of R¹ in a complex is conjugated to a different amino acid residue of the antibody. In some embodiments, each different amino acid comprises an F-amino group (e.g., lysine, arginine). However, in some embodiments, each different amino acid to which R¹ is covalently linked is a cysteine. In some embodiments, each different amino acid to which R¹ is covalently linked is a lysine. In some embodiments, R¹ is directly covalently linked to an amino acid residue of the antibody. However, in some embodiments, R¹ is indirectly covalently linked to an amino acid of the antibody, e.g., covalently linked to a glycosylation site on the amino acid. In some embodiments, R¹ is not covalently linked to an amino acid residue residing in a CDR region of the antibody.

In some embodiments, complexes disclosed herein comprise a structure of formula (I): [R¹]ₙ₁—R², in which each R¹ independently comprises a group of the formula (Ia):

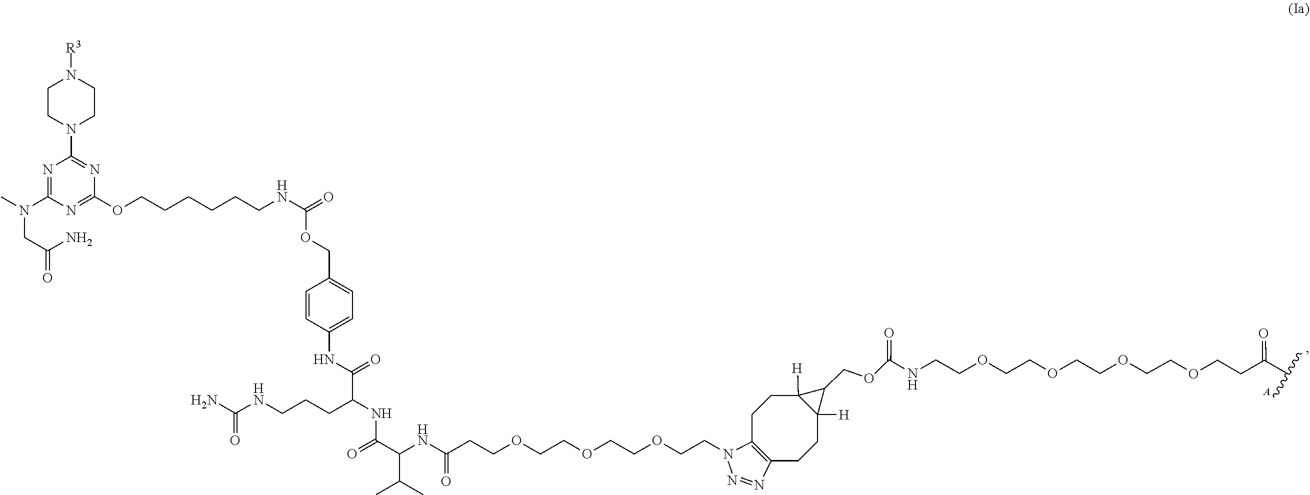

in which R³ is an oligonucleotide, e.g., a phosphorodiamidate morpholino oligomer (PMO); wherein in each complex n1 is independently an integer (e.g., of one or greater) representing the number of instances of R¹ in each complex, and each R¹ is covalently linked (e.g., indirectly or directly linked, e.g., directly linked) to R² at attachment point A. In some embodiments, R² comprises an anti-TfR1 antibody comprising a sequence as set forth in Table 2. For example, in some embodiments, R² comprises an anti-TfR1 antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody comprising a VH comprising the amino acid sequence of SEQ ID NO: 17 and/or comprising a VL comprising the amino acid sequence of SEQ ID NO: 18. In some embodiments, R² comprises an anti-TfR1 antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody comprising a heavy chain comprising the amino acid sequence of SEQ ID NO: 19 and/or comprising a light chain comprising the amino acid sequence of SEQ ID NO: 20. In some embodiments, R² comprises an anti-TfR1 antibody that is a Fab fragment, a full-length IgG, a Fab′ fragment, a F(ab′)₂ fragment, an scFv, or an Fv. In some embodiments, R² comprises an anti-TfR1 antibody that is a Fab fragment. In some embodiments, R³ is an oligonucleotide, e.g., a phosphorodiamidate morpholino oligomer (PMO) comprising the base sequence of CAATGCCATCCTGGAGTTCCTG (SEQ ID NO: 21). In some embodiments, each internucleoside linkage of the PMO is a phosphorodiamidate linkage. In some embodiments, R² comprises a Fab and each R¹ is covalently linked (e.g., indirectly or directly linked, e.g., directly linked) at attachment point A to a different amino acid residue of the Fab, optionally wherein each different amino acid residue is a lysine. In some embodiments, each R¹ is covalently linked to R² at attachment point A via a linkage site represented by a lysine (K) residue of the antibody. In some embodiments, a light chain constant region of an antibody of a complex is covalently linked to an oligonucleotide at a linkage site represented by K188 (based on Kabat numbering) and/or a linkage site represented by K190 (based on Kabat numbering) of the light chain constant region. In some embodiments, a light chain of an antibody of a complex is covalently linked to an oligonucleotide at a linkage site represented by a lysine (K) residue in a sequence motif DYEKHKVYA (SEQ ID NO: 27) of the light chain. In some embodiments, in each complex n1 is independently an integer (e.g., an integer in the range of 1-27, 1-26, 1-10, 1-5, or 1-3).

In some embodiments, complexes disclosed herein comprise a structure of formula (I): [R¹]ₙ₁—R², in which each R¹ comprises a group of the formula (Ib):

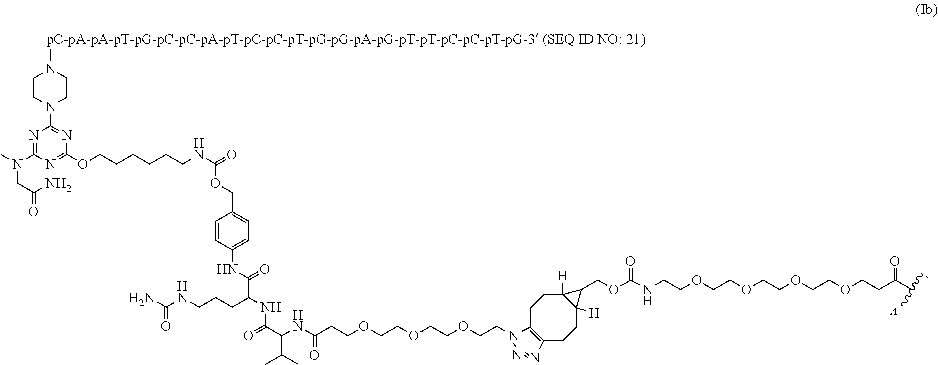

in which -p is a phosphorodiamidate linkage of a phosphorodiamidate morpholino oligomer (PMO), and wherein the PMO comprises a base sequence of CAATGCCATCCTGGAGTTCCTG (SEQ ID NO: 21); wherein in each complex n1 is independently an integer (e.g., of one or greater) representing the number of instances of R¹ in each complex, and each R¹ is covalently linked (e.g., indirectly or directly linked, e.g., directly linked) to R² at attachment point A. In some embodiments, R² comprises an anti-TfR1 antibody comprising a sequence as set forth in Table 2. For example, in some embodiments, R² comprises an anti-TfR1 antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody comprising a VH comprising the amino acid sequence of SEQ ID NO: 17 and/or comprising a VL comprising the amino acid sequence of SEQ ID NO: 18. In some embodiments, R² comprises an anti-TfR1 antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody comprising a heavy chain comprising the amino acid sequence of SEQ ID NO: 19 and/or comprising a light chain comprising the amino acid sequence of SEQ ID NO: 20. In some embodiments, R² comprises an anti-TfR1 antibody that is a Fab fragment, a full-length IgG, a Fab′ fragment, a F(ab′)₂ fragment, an scFv, or an Fv. In some embodiments, R² comprises an anti-TfR1 antibody that is a Fab fragment. In some embodiments, in each complex n1 is independently an integer (e.g., an integer in the range of 1-27, 1-26, 1-10, 1-5, or 1-3). In some embodiments, R² comprises a Fab and each R¹ is covalently linked (e.g., indirectly or directly linked, e.g., directly linked) at attachment point A to a different amino acid residue of the Fab, optionally wherein each different amino acid residue is a lysine. In some embodiments, each R¹ is covalently linked to R² at attachment point A via a linkage site represented by a lysine (K) residue of the antibody. In some embodiments, a lysine (K) residue linkage site of the anti-TfR1 antibody is represented by K188 (based on Kabat numbering) and/or K190 (based on Kabat numbering) of a light chain constant region of the anti-TfR1 antibody. In some embodiments, a lysine (K) residue linkage site is represented by a lysine (K) residue in a sequence motif DYEKHKVYA (SEQ ID NO: 27) of a light chain of the anti-TfR1 antibody.

In some embodiments, complexes disclosed herein comprise a structure of formula (I): [R¹]ₙ₁—R², in which each R¹ comprises a group of the formula (Ic):

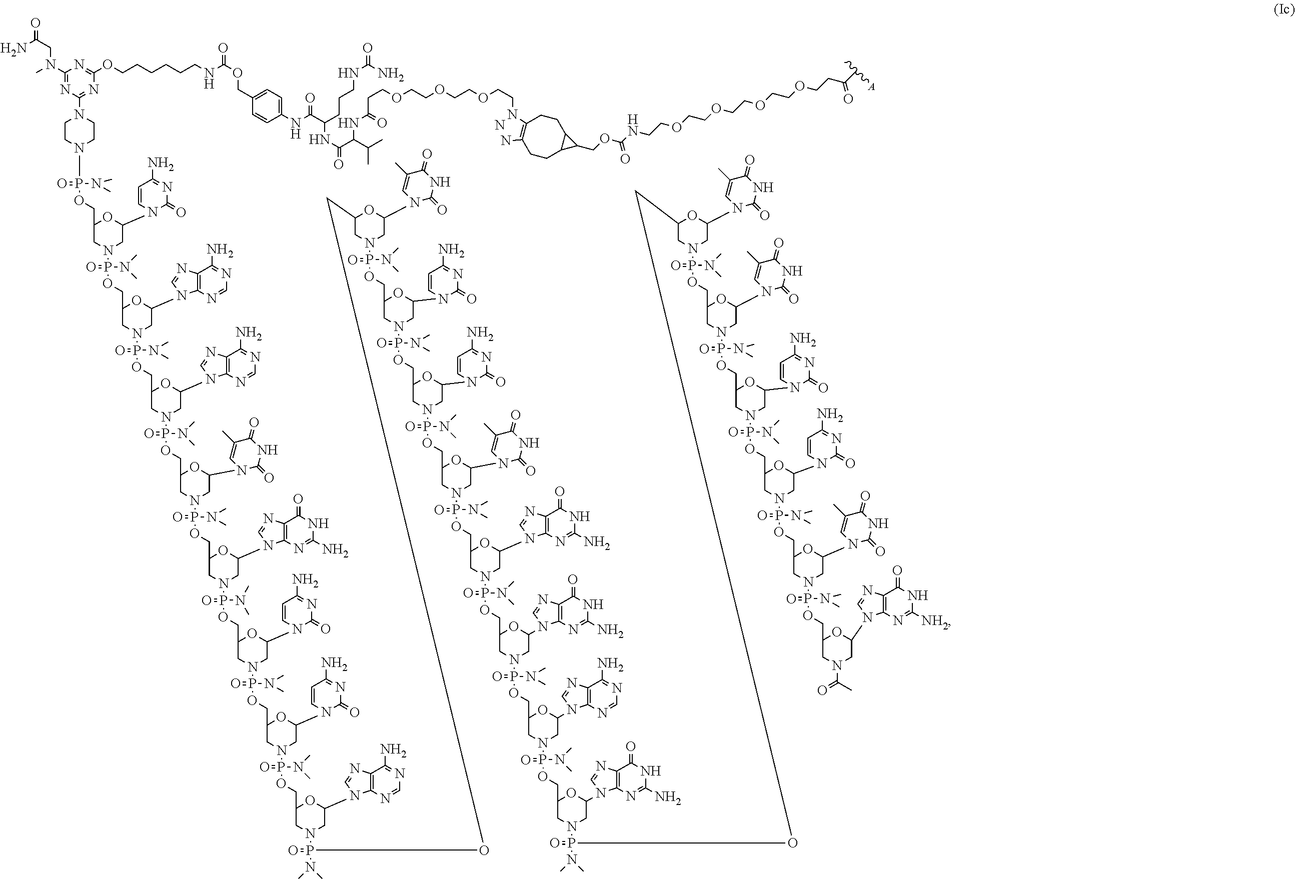

wherein in each complex n1 is independently an integer (e.g., of one or greater) representing the number of instances of R¹ in each complex, wherein each R¹ is covalently linked (e.g., indirectly or directly linked, e.g., directly linked) to R² at attachment point A. In some embodiments, R² comprises an anti-TfR1 antibody comprising a sequence as set forth in Table 2. For example, in some embodiments, R¹ comprises an anti-TfR1 antibody comprising a heavy chain complementarity determining region 1 (CDR-H1) comprising a sequence as set forth in SEQ ID NOs: 1, 7, or 12, a heavy chain complementarity determining region 2 (CDR-H-2) comprising a sequence as set forth in SEQ TD NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ TD NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody comprising a VH comprising the amino acid sequence of SEQ ID NO: 17 and/or comprising a VL comprising the amino acid sequence of SEQ ID NO: 18. In some embodiments, R² comprises an anti-TfR1 antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody comprising a heavy chain comprising the amino acid sequence of SEQ ID NO: 19 and/or comprising a light chain comprising the amino acid sequence of SEQ ID NO: 20. In some embodiments, R² comprises an anti-TfR1 antibody that is a Fab fragment, a full-length IgG, a Fab′ fragment, a F(ab′)₂ fragment, an scFv, or an Fv. In some embodiments, R² comprises an anti-TfR1 antibody that is a Fab fragment. In some embodiments, in each complex n1 is independently an integer (e.g., an integer in the range of 1-27, 1-26, 1-10, 1-5, or 1-3). In some embodiments, R² comprises a Fab and each R¹ is covalently linked (e.g., indirectly or directly linked, e.g., directly linked) at attachment point A to a different amino acid residue of the Fab, optionally wherein each different amino acid residue is a lysine. In some embodiments, each R¹ is covalently linked to R² at attachment point A via a linkage site represented by a lysine (K) residue of the antibody. In some embodiments, a lysine (K) residue linkage site of the anti-TfR1 antibody is represented by K188 (based on Kabat numbering) and/or K190 (based on Kabat numbering) of a light chain constant region of the anti-TfR1 antibody. In some embodiments, a lysine (K) residue linkage site is represented by a lysine (K) residue in a sequence motif DYEKHKVYA (SEQ ID NO: 27) of a light chain of the anti-TfR1 antibody.

In some embodiments, complexes disclosed herein comprise a structure of formula (Id):

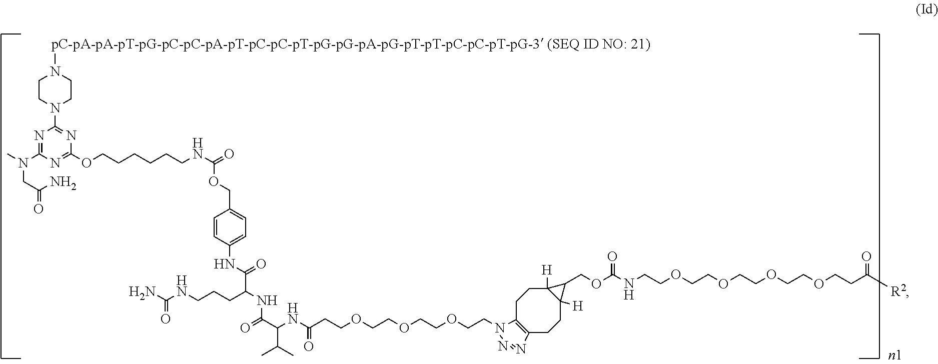

in which -p is a phosphorodiamidate linkage of a phosphorodiamidate morpholino oligomer (PMO), and wherein the PMO comprises a base sequence of CAATGCCATCCTGGAGTTCCTG (SEQ ID NO: 21); wherein R² comprises an anti-TfR11 antibody (e.g., a Fab) comprising a CDR-H1, a CDR-H2, a CDR-H3, a CDR-L1, a CDR-L2, and a CDR-L3 selected from Table 2, optionally wherein the antibody (e.g., a Fab) comprises a VH comprising the amino acid sequence of SEQ ID NO: 17 and a VL comprising the amino acid sequence of SEQ ID NO: 18, further optionally wherein the antibody (e.g., a Fab) comprises a heavy chain comprising the amino acid sequence of SEQ ID NO: 19 and a light chain comprising the amino acid sequence of SEQ ID NO: 20; and wherein in each complex n1 is independently an integer (e.g., of one or greater) representing the number of instances of the group enclosed by square brackets, wherein each instance of the group enclosed by square brackets is covalently linked to a different amino acid residue of the antibody (e.g. a Fab), optionally wherein each different amino acid residue is a lysine. In some embodiments, R² comprises an anti-TfR1 antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody (e.g., a Fab) comprising a VH comprising the amino acid sequence of SEQ ID NO: 17 and/or comprising a VL comprising the amino acid sequence of SEQ ID NO: 18. In some embodiments, R² comprises an anti-TfR1 antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody (e.g., a Fab) comprising a heavy chain comprising the amino acid sequence of SEQ ID NO: 19 and/or comprising a light chain comprising the amino acid sequence of SEQ ID NO: 20. In some embodiments, in each complex n1 is independently an integer (e.g., an integer in the range of 1-27, 1-26, 1-10, 1-5, or 1-3). In some embodiments, R² comprises an anti-TfR1 antibody (e.g., a Fab) that is covalently linked (e.g., indirectly or directly linked, e.g., directly linked) via different amino acid residue of the antibody (e.g., Fab), optionally wherein each different amino acid residue is a lysine. In some embodiments, each instance of the group enclosed by square brackets in the structure of formula (Id) is covalently linked to a linkage site represented by a lysine (K) residue of the anti-TfR1 antibody (e.g., the Fab). In some embodiments, a lysine (K) residue linkage site of the anti-TfR1 antibody is represented by K188 (based on Kabat numbering) and/or K190 (based on Kabat numbering) of a light chain constant region of the anti-TfR1 antibody. In some embodiments, a lysine (K) residue linkage site is represented by a lysine (K) residue in a sequence motif DYEKHKVYA (SEQ ID NO: 27) of a light chain of the anti-TfR1 antibody.

In some embodiments, complexes described herein comprise a structure of formula (A):

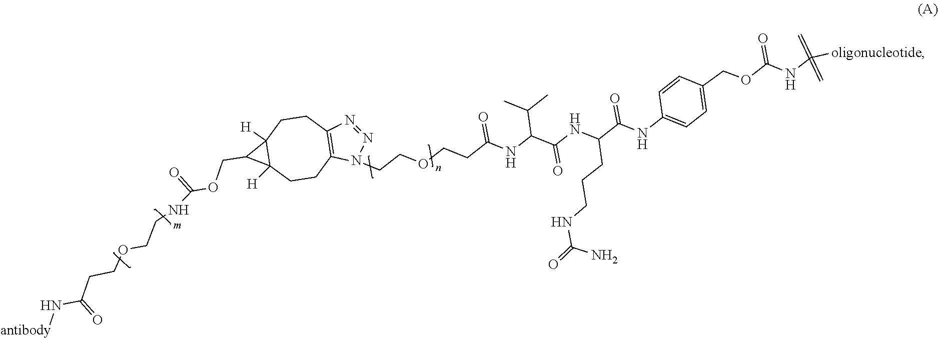

wherein n is 0-15 (e.g., 3) and m is 0-15 (e.g., 4). In some embodiments, the antibody is an anti-TfR1 antibody (e.g., the anti-TfR1 antibody provided in Table 2). In some embodiments, the oligonucleotide is a PMO and comprises the nucleotide sequence of SEQ ID NO: 21. In some embodiments, each internucleoside linkage of the PMO is a phosphorodiamidate linkage. In some embodiments, the amide shown adjacent to the anti-TfR1 antibody in the structure results from a reaction with an amine of the anti-TfR1 antibody, such as a lysine epsilon amine. In some embodiments, a complex described herein comprises an anti-TfR1 Fab covalently linked via a lysine of the Fab to the 5′ end of a PMO. In some embodiments, the antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the antibody comprises a VH comprising the amino acid sequence of SEQ ID NO: 17 and/or comprises a VL comprising the amino acid sequence of SEQ ID NO: 18. In some embodiments, the antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)₂ fragment, an scFv, or an Fv.

In each complex disclosed (e.g., comprising a structure of formula (I): [R¹]ₙ₁—R², such as a complex in which each R¹ comprises a group of the formula (Ia), (Ib), or (Ic); a complex comprising a structure of formula (Id); or a complex comprising a structure of formula (A)) may comprise a structure having the stereochemistry shown in formula (B):

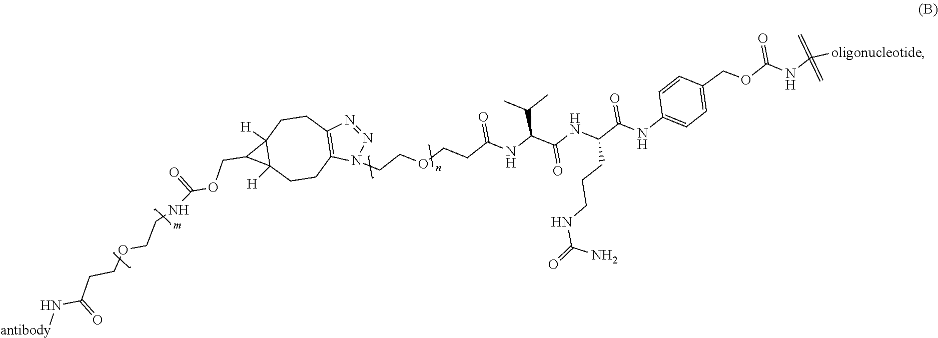

wherein n is 0-15 (e.g., 3) and m is 0-15 (e.g., 4). It should be understood that the stereochemistry shown in formula (B) can be applied to the corresponding portion of any formula or structure provided herein (e.g., formula (Ia), (Ib), (Ic), (Id), or (A)).

In some embodiments, a light chain constant region of an antibody of a complex disclosed herein (e.g., a complex comprising a structure of formula (I): [R¹]ₙ₁—R², such as one in which each R¹ comprises a group of the formula (Ia), (Ib), or (Ic); a complex comprising a structure of formula (Id); or a complex comprising a structure of formula (A)) is independently covalently linked to an oligonucleotide at a linkage site represented by K188 (based on Kabat numbering) and/or a linkage site represented by K190 (based on Kabat numbering) of the light chain constant region. In some embodiments, a light chain of an antibody of a complex disclosed herein (e.g., a complex comprising a structure of formula (I): [R¹]ₙ₁—R², such as one in which each R¹ comprises a group of the formula (Ia), (Ib), or (Ic); a complex comprising a structure of formula (Id); or a complex comprising a structure of formula (A)) is independently covalently linked to an oligonucleotide at a linkage site represented by a lysine (K) residue in a sequence motif DYEKHIKVYA (SEQ ID NO: 27) of the light chain.

### Antibodies

In some embodiments, complexes described herein comprise an antibody that binds human transferrin receptor 1 (TfR1). An example human TfR1 amino acid sequence, corresponding to NCBI sequence NP_003225.2 (transferrin receptor protein 1 isoform 1, Homo sapiens) is as follows:

(SEQ ID NO: 22)
MMDQARSAFSNLFGGEPLSYTRFSLARQVDGDNSHVEMKLAVDEEE
NADNNTKANVTKPKRCSGSICYGTIAVIVFFLIGFMIGYLGYCKG
VEPKTECERLAGTESPVREEPGEDFPAARRLYWDDLKRKLSEKLD
STDFTGTIKLLNENSYVPREAGSQKDENLALYVENQFREFKLSKV
WRDQHFVKIQVKDSAQNSVIIVDKNGRLVYLVENPGGYVAYSKAA
TVTGKLVHANFGTKKDFEDLYTPVNGSIVIVRAGKITFAEKVANA
ESLNAIGVLIYMDQTKFPIVNAELSFFGHAHLGTGDPYTPGFPSF
NHTQFPPSRSSGLPNIPVQTISRAAAEKLFGNMEGDCPSDWKTDS
TCRMVTSESKNVKLTVSNVLKEIKILNIFGVIKGFVEPDHYVVVG
AQRDAWGPGAAKSGVGTALLLKLAQMFSDMVLKDGFQPSRSIIFA
SWSAGDFGSVGATEWLEGYLSSLHLKAFTYINLDKAVLGTSNFKV
SASPLLYTLIEKTMQNVKHPVTGQFLYQDSNWASKVEKLTLDNAA
FPFLAYSGIPAVSFCFCEDTDYPYLGTTMDTYKELIERIPELNKV
ARAAAEVAGQFVIKLTHDVELNLDYERYNSQLLSFVRDLNQYRAD
IKEMGLSLQWLYSARGDFFRATSRLTTDFGNAEKTDRFVMKKLND
RVMRVEYHFLSPYVSPKESPFRHVFWGSGSHTLPALLENLKLRKQ
NNGAFNETLFRNQLALATWTIQGAANALSGDVWDIDNEF.

Table 2 provides examples of sequences of an anti-TfR1 antibody useful in the complexes provided herein.

TABLE 2
Examples of anti-TfR1 antibody sequences

| Feature | IMGT | Kabat | Chothia |
|---|---|---|---|
| CDR-H1 | GYSITSGYY (SEQ ID | SGYYWN (SEQ ID | GYSITSGY (SEQ ID |
| | NO: 1) | NO: 7) | NO: 12) |
| CDR-H2 | ITFDGAN (SEQ ID | YITFDGANNYNPSLK | FDG (SEQ ID NO: 13) |
| | NO: 2) | N (SEQ ID NO: 8) | |
| CDR-H3 | TRSSYDYDVLDY | SSYDYDVLDY (SEQ | SYDYDVLD (SEQ ID |
| | (SEQ ID NO: 3) | ID NO: 9) | NO: 14) |
| CDR-L1 | QDISNF | RASQDISNFLN (SEQ | SQDISNF (SEQ ID NO: |
| | (SEQ ID NO: 4) | ID NO: 10) | 15) |
| CDR-L2 | YTS (SEQ ID NO: 5) | YTSRLHS (SEQ ID | YTS (SEQ ID NO: 5) |
| | | NO: 11) | |
| CDR-L3 | QQGHTLPYT (SEQ ID | QQGHTLPYT (SEQ ID | GHTLPY (SEQ ID NO: |
| | NO: 6) | NO: 6) | 16) |

| | |
|---|---|
| VH | QVQLQESGPGLVKPSQTLSLTCTVTGYSITSGYYWNWIRQPPGKGLEWI |
| | GYITFDGANNYNPSLKNRVSISRDTSKNQFSLKLSSVTAEDTATYYCTR |
| | SSYDYDVLDYWGQGTTVTVSS (SEQ ID NO: 17) |
| VL | DIQMTQSPSSLSASVGDRVTITCRASQDISNFLNWYQQKPGQPVKLLIY |
| | YTSRLHSGVPSRFSGSGSGTDFTLTISSLQPEDFATYYCQQGHTLPYTFG |
| | QGTKLEIK (SEQ ID NO: 18) |
| Fab HC | QVQLQESGPGLVKPSQTLSLTCTVTGYSITSGYYWNWIRQPPGKGLEWI |
| | GYITFDGANNYNPSLKNRVSISRDTSKNQFSLKLSSVTAEDTATYYCTR |
| | SSYDYDVLDYWGQGTTVTVSSASTKGPSVFPLAPSSKSTSGGTAALGC |
| | LVKDYFPEPVTVSWNSGALTSGVHTFPAVLQSSGLYSLSSVVTVPSSSL |
| | GTQTYICNVNHKPSNTKVDKKVEPKSCDKTHT (SEQ ID NO: 19) |
| Fab LC | DIQMTQSPSSLSASVGDRVTITCRASQDISNFLNWYQQKPGQPVKLLIY |
| | YTSRLHSGVPSRFSGSGSGTDFTLTISSLQPEDFATYYCQQGHTLPYTFG |
| | QGTKLEIKRTVAAPSVFIFPPSDEQLKSGTASVVCLLNNFYPREAKVQW |
| | KVDNALQSGNSQESVTEQDSKDSTYSLSSTLTLSKADYEKHKVYACEV |
| | THQGLSSPVTKSFNRGEC (SEQ ID NO: 20) |

In some embodiments, the anti-TfR1 antibody of the present disclosure comprises a heavy chain complementarity determining region 1 (CDR-H1) of SEQ ID NO: 1 (according to the IMGT definition system), a heavy chain complementarity determining region 2 (CDR-H2) of SEQ ID NO: 2 (according to the IMGT definition system), a heavy chain complementarity determining region 3 (CDR-H3) of SEQ ID NO: 3 (according to the IMGT definition system), a light chain complementarity determining region 1 (CDR-L1) of SEQ ID NO: 4 (according to the IMGT definition system), a light chain complementarity determining region 2 (CDR-L2) of SEQ ID NO: 5 (according to the IMGT definition system), and a light chain complementarity determining region 3 (CDR-L3) of SEQ ID NO: 6 (according to the IMGT definition system).

In some embodiments, the anti-TfR1 antibody of the present disclosure comprises a heavy chain complementarity determining region 1 (CDR-H1) of SEQ ID NO: 7 (according to the Kabat definition system), a heavy chain complementarity determining region 2 (CDR-H2) of SEQ ID NO: 8 (according to the Kabat definition system), a heavy chain complementarity determining region 3 (CDR-H3) of SEQ ID NO: 9 (according to the Kabat definition system), a light chain complementarity determining region 1 (CDR-L1) of SEQ ID NO: 10 (according to the Kabat definition system), a light chain complementarity determining region 2 (CDR-L2) of SEQ ID NO: 11 (according to the Kabat definition system), and a light chain complementarity determining region 3 (CDR-L3) of SEQ ID NO: 6 (according to the Kabat definition system).

In some embodiments, the anti-TfR1 antibody of the present disclosure comprises a heavy chain complementarity determining region 1 (CDR-H1) of SEQ ID NO: 12 (according to the Chothia definition system), a heavy chain complementarity determining region 2 (CDR-H2) of SEQ ID NO: 13 (according to the Chothia definition system), a heavy chain complementarity determining region 3 (CDR-H3) of SEQ ID NO: 14 (according to the Chothia definition system), a light chain complementarity determining region 1 (CDR-L1) of SEQ ID NO: 15 (according to the Chothia definition system), a light chain complementarity determining region 2 (CDR-L2) of SEQ ID NO: 5 (according to the Chothia definition system), and a light chain complementarity determining region 3 (CDR-L3) of SEQ ID NO: 16 (according to the Chothia definition system).

In some embodiments, the anti-TfR1 antibody of the present disclosure comprises a heavy chain variable region (VH) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH comprising the amino acid sequence of SEQ ID NO: 17. Alternatively or in addition (e.g., in addition), the anti-TfR1 antibody of the present disclosure comprises a light chain variable region (VL) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL comprising the amino acid sequence of SEQ ID NO: 18.

In some embodiments, the anti-TfR1 antibody of the present disclosure comprises a VH comprising an amino acid sequence that is at least 75% (e.g., 75%, 80%, 85%, 90%, 95%, 98%, or 99%) identical in the framework regions to the VH comprising the amino acid sequence of SEQ ID NO: 17. Alternatively or in addition (e.g., in addition), in some embodiments, the anti-TfR1 antibody of the present disclosure comprises a VL comprising an amino acid sequence that is at least 75% (e.g., 75%, 80%, 85%, 90%, 95%, 98%, or 99%) identical in the framework regions to the VL comprising the amino acid sequence of SEQ ID NO: 18.

In some embodiments, the anti-TfR1 antibody of the present disclosure comprises a VH comprising the amino acid sequence of SEQ ID NO: 17. Alternatively or in addition (e.g., in addition), in some embodiments, the anti-TfR1 antibody of the present disclosure comprises a VL comprising the amino acid sequence of SEQ ID NO: 18.

In some embodiments, the anti-TfR1 antibody of the present disclosure comprises a heavy chain comprising an amino acid sequence least 75% (e.g., 75%, 80%, 85%, 90%, 95%, 98%, or 99%) identical to the amino acid sequence of SEQ ID NO: 19. Alternatively or in addition (e.g., in addition), the anti-TfR1 antibody of the present disclosure comprises a light chain comprising an amino acid sequence least 75% (e.g., 75%, 80%, 85%, 90%, 95%, 98%, or 99%) identical to the amino acid sequence of SEQ ID NO: 20. In some embodiments, the anti-TfR1 antibody of the present disclosure is a Fab that comprises a heavy chain comprising an amino acid sequence least 75% (e.g., 75%, 80%, 85%, 90%, 95%, 98%, or 99%) identical to the amino acid sequence of SEQ ID NO: 19. Alternatively or in addition (e.g., in addition), the anti-TfR1 antibody of the present disclosure is a Fab that comprises a light chain comprising an amino acid sequence least 75% (e.g., 75%, 80%, 85%, 90%, 95%, 98%, or 99%) identical to the amino acid sequence of SEQ ID NO: 20.

In some embodiments, the anti-TfR1 antibody of the present disclosure comprises a heavy chain comprising the amino acid sequence of SEQ ID NO: 19. Alternatively or in addition (e.g., in addition), the anti-TfR1 antibody of the present disclosure comprises a light chain comprising the amino acid sequence of SEQ ID NO: 20. In some embodiments, the anti-TfR1 antibody of the present disclosure is a Fab that comprises a heavy chain comprising an amino acid sequence of SEQ ID NO: 19. Alternatively or in addition (e.g., in addition), the anti-TfR1 antibody of the present disclosure is a Fab that comprises a light chain comprising the amino acid sequence of SEQ ID NO: 20.

In some embodiments, the anti-TfR1 antibody provided herein may have one or more post-translational modifications. In some embodiments, N-terminal cyclization, also called pyroglutamate formation (pyro-Glu), may occur in the antibody at N-terminal Glutamate (Glu) and/or Glutamine (Gln) residues during production. As such, it should be appreciated that an antibody specified as having a sequence comprising an N-terminal glutamate or glutamine residue encompasses antibodies that have undergone pyroglutamate formation resulting from a post-translational modification. In some embodiments, pyroglutamate formation occurs in a heavy chain sequence. In some embodiments, pyroglutamate formation occurs in a light chain sequence.

### Linkage Sites

Provided herein are complexes comprising an antibody covalently linked to one or more oligonucleotides. In some embodiments, the antibody comprises a heavy chain comprising a heavy chain variable region (VH) and a heavy chain constant region, and a light chain comprising a light chain variable region (VL) and a light chain constant region. In some embodiments, each of the one or more oligonucleotides is covalently linked at a linkage site represented by a lysine (K) residue of the antibody. In some embodiments, provided herein are compositions comprising a plurality of complexes, wherein each complex comprises an antibody covalently linked to one or more oligonucleotides, wherein each of the one or more oligonucleotides is linked to the antibody via a distinct linkage site.

In some embodiments, a linkage site is in the light chain of the antibody. In some embodiments, a linkage site is in the light chain constant region of the antibody. For example, in some embodiments, a linkage site is represented by K188 or K190 of the light chain constant region based on Kabat numbering.

In some embodiments, light chain constant regions of antibodies of complexes in a composition are independently covalently linked to an oligonucleotide at a linkage site represented by K188 (based on Kabat numbering) and/or a linkage site represented by K190 (based on Kabat numbering) of a light chain constant region of an antibody. For example, in some embodiments, a linkage site is represented by K188 (based on Kabat numbering) of a light chain constant region of an antibody. In some embodiments, a linkage site is represented by K190 (based on Kabat numbering) of a light chain constant region of an antibody. In some embodiments, linkage sites are represented by K188 (based on Kabat numbering) and K190 (based on Kabat numbering) of a light chain constant region of an antibody. In some embodiments, at least 80% (e.g., at least 80%, at least 81%, at least 82%, at least 83%, at least 84%, at least 85% at least 86%, at least 87%, at least 88%, at least 89%, at least 90%, at least 91%, at least 92%, at least 93%, at least 94%, at least 95%, a least 96%, at least 97%, at least 98%, or at least 99%) of the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant regions of the antibodies. In some embodiments, about 80%-98%, 80%-95%, 80%-90%, 80%-85%, 85%-98%, 85%-95%, 85%-90%, 90%-98%, 90%-95%, 95-97%, or 95%-98% of the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant regions of the antibodies. In some embodiments, about 85%-95% (e.g., 85%-95%, 85%-90%, or 90%-95%) of the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant regions of the antibodies. In some embodiments, 90%-95% (e.g. about 90%, about 91%, about 92%, about 93%, about 94%, or about 95%) of the light chain constant regions of the antibodies of the complexes in the composition are independently covalently linked to an oligonucleotide at a linkage site represented by K188 (based on Kabat numbering) and/or a linkage site represented by K190 (based on Kabat numbering) of the light chain constant regions of the antibodies. It is to be understood that, complexes comprising light chain constant regions of antibodies covalently linked to an oligonucleotide at a linkage site represented by K188 (based on Kabat numbering) and/or a linkage site represented by K190 (based on Kabat numbering) of a light chain constant region include: complexes comprising antibodies that are covalently linked to an oligonucleotide at a linkage site represented by K188 (based on Kabat numbering) of the light chain constant regions of the antibodies; complexes comprising antibodies that are covalently linked to an oligonucleotide at a linkage site represented by K190 (based on Kabat numbering) of the light chain constant regions of the antibodies; and/or complexes comprising antibodies that are covalently linked to an oligonucleotide at a linkage site represented by K188 (based on Kabat numbering) and at a linkage site represented by K190 (based on Kabat numbering) of the light chain constant regions of the antibodies.

In some embodiments, lysine (K) residue numbers referred to herein are based on Kabat numbering (Kabat et al. (1971) Ann. NY Acad, Sci. 190:382-391 and Kabat et al., Sequences of Proteins of Immunological Interest (National Institutes of Health, Bethesda, Md. (1987) and (1991)). The variable regions and constant regions of the heavy and light chains of the antibodies provided herein are numbered separately. Kabat numbering of the light chain variable regions and heavy chain variable regions of the antibodies are described in the art, e.g., in Kabat et al. (1971) Ann. NY Acad, Sci. 190:382-391 and Kabat et al., Sequences of Proteins of Immunological Interest (National Institutes of Health, Bethesda, Md. (1987) and (1991). Kabat numbering of the light chain constant regions and heavy chain constant regions of the antibodies provided herein can be found at imgt.org/IMGTScientificChart/Numbering/Hu_IGHGnber.html and imgt.org/IMGTScientificChart/Numbering/Hu_IGKCnber.html (also see Edelman, G. M. et al., Proc. Natl. Acad. USA, 63, 78-85 (1969). PMID: 5257969; Hieter, P. A. et al., Cell, 22, 197-207 (1980). PMID: 6775818; Sequences of Proteins of Immunological Interest (National Institutes of Health, Bethesda, Md. 647, 662, 680, 689 (1991)).

In some embodiments, light chains of antibodies of complexes in a composition are independently covalently linked to an oligonucleotide at linkage sites represented by lysine (K) residues in a sequence motif DYEKHKVYA (SEQ ID NO: 27) of light chains of the antibodies. For example, in some embodiments, a linkage site is represented by the K at position 4 of a sequence motif DYEKHKVYA (SEQ ID NO: 27) of a light chain of an antibody. In some embodiments, a linkage site is represented by the K at position 6 of a sequence motif DYEKHKVYA (SEQ ID NO: 27) of a light chain of an antibody. In some embodiments, linkage sites are represented by the K at position 4 and the K at position 6 of a sequence motif DYEKHKVYA (SEQ ID NO: 27) of a light chain of an antibody. In some embodiments, at least 80% (e.g., at least 80%, at least 81%, at least 82%, at least 83%, at least 84%, at least 85% at least 86%, at least 87%, at least 88%, at least 89%, at least 90%, at least 91%, at least 92%, at least 93%, at least 94%, at least 95%, a least 96%, at least 97%, at least 98%, or at least 99%) of the light chains of the antibodies of the complexes in the composition are independently covalently linked to an oligonucleotide at linkage sites represented by lysine (K) residues in a sequence motif DYEKHKVYA (SEQ ID NO: 27) of the light chains of the antibodies. In some embodiments, 80%-98%, 80%-95%, 80%-90%, 80%-85%, 85%-98%, 85%-95, 85%-90%, 90%-98%, 90%-95%, 95%-97%, or 95%-98% of the light chains of the antibodies of the complexes in the composition are independently covalently linked to an oligonucleotide at linkage sites represented by lysine (K) residues in a sequence motif DYEKHKVYA (SEQ ID NO: 27) of the light chains of the antibodies. In some embodiments, 85%-95% (e.g., 85%-95%, 85%-90%, or 90%-95%) of the light chains of the antibodies of the complexes in the composition are independently covalently linked to an oligonucleotide at linkage sites represented by lysine (K) residues in a sequence motif DYEKHKVYA (SEQ ID NO: 27) of the light chains of the antibodies. In some embodiments, 90%-95% (e.g., about 90%, about 91%, about 92%, about 93%, about 94%, or about 95%) of the light chains of the antibodies of the complexes in the composition are independently covalently linked to an oligonucleotide at linkage sites represented by lysine (K) residues in a sequence motif DYEKHKVYA (SEQ ID NO: 27) of the light chains of the antibodies. It is to be understood that, complexes comprising light chains of antibodies covalently linked to an oligonucleotide at a linkage site represented by lysine (K) residues in a sequence motif DYEKHKVYA (SEQ ID NO: 27) of the light chains of the antibodies include: complexes comprising antibodies that are covalently linked to an oligonucleotide at a linkage site represented by the K at position 4 in a sequence motif DYEKHKVYA (SEQ ID NO: 27) of the light chains of the antibodies; complexes comprising antibodies that are covalently linked to an oligonucleotide at a linkage site represented by the K at position 6 in a sequence motif DYEKHKVYA (SEQ ID NO: 27) of the light chains of the antibodies; and/or complexes comprising antibodies that are covalently linked to an oligonucleotide at a linkage site represented the K at position 4 and at a linkage site represented by the K at position 6 in a sequence motif DYEKHKVYA (SEQ ID NO: 27) of the light chains.

The term “about” as used herein, refers to a ±5%-10% of variation based on the % the term is used to modify.

### Oligonucleotides

In some embodiments, an oligonucleotide of a complex described herein is a single stranded oligonucleotide. In some embodiments, the oligonucleotide is useful for targeting DMD (e.g., for exon skipping). In some embodiments, an oligonucleotide that is useful for targeting DMD (e.g., for exon skipping) targets a DMD allele (e.g., a mutated DMD allele). In some embodiments, an oligonucleotide useful for targeting DMD (e.g., for exon skipping) targets a region of a DMD RNA (e.g., the Dp427m transcript of SEQ ID NO: 23, or a DMD RNA, such as a pre-mRNA, comprising a sequence of SEQ ID NO: 26). In some embodiments, an oligonucleotide useful for targeting DMD (e.g., for exon skipping) comprises a region of complementarity to a DMD RNA (e.g., the Dp427m transcript of SEQ ID NO: 23). In some embodiments, an oligonucleotide useful for targeting DMD (e.g., for exon skipping) comprises a region of complementarity to an exon (e.g., exons 45) or an intron of a DMD RNA. In some embodiments, an oligonucleotide useful for targeting DMD (e.g., for exon skipping) targets a splice donor site, a splice acceptor site, a branch point, or an exonic splicing enhancer (ESE) of a DMD RNA (e.g., a DMD pre-mRNA encoded by Homo sapiens dystrophin (DMD) gene (e.g., NCBI Accession No. NG_012232.1). In some embodiments, an oligonucleotide useful for targeting DMD (e.g., for exon skipping) targets an exonic splicing enhancer (ESE) sequence in DMD (e.g., an ESE sequence of exon 45). In some embodiments, an oligonucleotide useful for targeting DMD (e.g., for exon skipping) targets a splice acceptor site sequence in DMD (e.g., a splice acceptor site sequence of exon 45).

Examples of DMD RNA sequences and exon sequences that may be targeted by an oligonucleotide of a complex are provided below.

Homo sapiens dystrophin (DMD), transcript variant Dp427m, mRNA (NCBI Reference Sequence: NM_004006.2; SEQ ID NO: 23).

Homo sapiens dystrophin (DMD), locus around exon 45 (nucleotide positions 1376066 to 1376301 of NCBI Reference Sequence: NG_012232.1; exon 45 is underlined):

(SEQ ID NO: 26)
TTTTTGTTTTGCCTTTTTGGTATCTTACAGGAACTCCAGGATGGC
ATTGGGCAGCGGCAAACTGTTGTCAGAACATTGAATGCAACTGGG
GAAGAAATAATTCAGCAATCCTCAAAAACAGATGCCAGTATTCTA
CAGGAAAAATTGGGAAGCCTGAATCTGCGGTGGCAGGAGGTCTGC
AAACAGCTGTCAGACAGAAAAAAGAGGTAGGGCGACAGATCTAAT
AGGAATGAAAA

Homo sapiens dystrophin (DMD), transcript variant Dp427m, exon 45 (nucleotide positions 6683-6858 of NCBI Reference Sequence: NM_004006.2):

(SEQ ID NO: 24)
GAACTCCAGGATGGCATTGGGCAGCGGCAAACTGTTGTCAGAACA
TTGAATGCAACTGGGGAAGAAATAATTCAGCAATCCTCAAAAACA
GATGCCAGTATTCTACAGGAAAAATTGGGAAGCCTGAATCTGCGG
TGGCAGGAGGTCTGCAAACAGCTGTCAGACAGAAAAAAGAG

In some embodiments, an oligonucleotide useful for targeting DMD (e.g., for exon skipping) is 10⁻³⁵ (e.g., 10-35, 15-35, 10-30, 15-30, 15-25, 17-27, 18-26, 19-25, 20-24, 20-35, 20-30, 20-25, 20-23, 21-24, 21-23, or 20-22) nucleotides in length. In some embodiments, an oligonucleotide useful for targeting DMD (e.g., for exon skipping) is 15, 16, 17, 18, 19, 20, 21, 22, 23, 24, 25, 26, 27, 28, 29, or 30 nucleotides in length, optionally 17-27, or 22 nucleotides in length.

In some embodiments, an oligonucleotide useful for targeting DMD (e.g., for exon skipping) comprises a region of complementarity of at least 8 (e.g., at least 8, 9, 10, 11, 12, 13, 14, 15, 16, 17, 18, 19, 20, 21, 22, 23, 24, 25, 26, 27, 28, 29, or 30) consecutive nucleotides to a DMD RNA. In some embodiments, an oligonucleotide useful for targeting DMD (e.g., for exon skipping) comprises a region of complementarity of at least 8 (e.g., at least 8, 9, 10, 11, 12, 13, 14, 15, 16, 17, 18, 19, 20, 21, 22, 23, 24, 25, 26, 27, 28, 29, or 30) consecutive nucleotides to an exon of a DMD RNA.

In some embodiments, an oligonucleotide useful for targeting DMD (e.g., for exon skipping) comprises a region of complementarity of at least 8 (e.g., at least 8, 9, 10, 11, 12, 13, 14, 15, 16, 17, 18, 19, 20, 21, 22, 23, 24, 25, 26, 27, 28, 29, or 30) consecutive nucleotides to a DMD sequence as set forth in any one of SEQ ID NOs: 23-26.

In some embodiments, an oligonucleotide useful for targeting DMD (e.g., for exon skipping) comprises a region of complementarity of at least 6 (e.g., at least 6, 7, 8, 9, 10, 11, 12, 13, 14, 15, 16, 17, 18, 19, 20, 21, or 22) consecutive nucleotides to a target sequence as set forth in SEQ ID NO: 25 (CAGGAACTCCAGGATGGCATTG). In some embodiments, an oligonucleotide useful for targeting DMD (e.g., for exon skipping) comprises at least 6 (e.g., at least 6, 7, 8, 9, 10, 11, 12, 13, 14, 15, 16, 17, 18, 19, 20, 21, or 22) consecutive nucleotides of a sequence as set forth in SEQ ID NO: 21 (CAATGCCATCCTGGAGTTCCTG).

In some embodiments, an oligonucleotide useful for targeting DMD (e.g., for exon skipping) comprises the nucleotide sequence of SEQ ID NO: 21. In some embodiments, any one of the oligonucleotides provided herein is a PMO. In some embodiments, each internucleoside linkage of the PMO is a phosphorodiamidate linkage.

In some embodiments, it should be appreciated that methylation of the nucleobase uracil at the C5 position forms thymine. Thus, in some embodiments, a nucleotide or nucleoside having a C5 methylated uracil (or 5-methyl-uracil) may be equivalently identified as a thymine nucleotide or nucleoside.

In some embodiments, any one or more of the thymine bases (T's) in any one of the oligonucleotides provided herein (e.g., the oligonucleotide as set forth in SEQ ID NO: 21) may independently and optionally be uracil bases (U's), and/or any one or more of the U's in the oligonucleotides provided herein may independently and optionally be T's.

### Compositions

In some embodiments, compositions comprising complexes (i.e., a plurality of complexes) are formulated in a manner suitable for the methods described herein. In some embodiments, compositions comprising muscle-targeting complexes are delivered to a subject using a formulation that minimizes degradation, facilitates delivery and/or (e.g., and) uptake, or provides another beneficial property to the complexes in the formulation. Accordingly, in some embodiments, compositions comprising complexes (e.g., a plurality of complexes comprising a PMO covalently linked with a Fab) are formulated with a suitable buffer (e.g., a pharmaceutical buffer). In some embodiments, compositions comprising muscle-targeting complexes (e.g., complexes comprising a PMO covalently linked with a Fab) are formulated in aqueous solutions. In some embodiments, compositions comprising a plurality of the complexes can be lyophilized (e.g., for storage). In some embodiments, the lyophilized composition may be reconstituted (e.g., with water) for administration to a subject. The compositions (e.g., in aqueous solutions or in lyophilized compositions) can be suitably prepared such that when administered to a subject, either into the immediate environment of a target cell or systemically, a sufficient amount of the complexes enter target muscle cells.

In some embodiments, compositions (e.g., in aqueous solutions) for administration to a subject in the methods described herein comprise complexes (i.e., a plurality of complexes), each of which complex comprises a phosphorodiamidate morpholino oligomer (PMO) covalently linked to an antibody. In some embodiments, compositions (e.g., in aqueous solutions) for administration to a subject in the methods described herein comprise complexes, in which each complex comprises a phosphorodiamidate morpholino oligomer (PMO) covalently linked to an anti-TfR1 antibody, optionally wherein the antibody of such complexes comprises a CDR-H1, CDR-H2, CDR-H3, CDR-L1, CDR-L2, and CDR-L3 as set forth in Table 2, further optionally wherein the antibody of such complexes comprises a VH and/or (e.g., and) a VL as set forth in Table 2. In some embodiments, the antibody is an anti-TfR1 Fab (e.g., an anti-TfR1 Fab comprising CDRs having amino acid sequences as set forth in Table 2, and/or having a VH and/or a VL having amino acid sequences as set forth in Table 2).

In some embodiments, compositions (e.g., in aqueous solutions) for administration to a subject in the methods described herein comprise complexes (i.e., a plurality of complexes) wherein each complex is a structure of the formula (I): [R¹]ₙ₁—R², in which each R¹ independently comprises a compound comprising an oligonucleotide (e.g., a PMO) and is covalently linked to R², wherein R² comprises an anti-TfR1 antibody, and in which in each complex n1 is independently an integer of one or greater representing the number of instances of R¹ in each complex.

In some embodiments, the value of n1 of each complex in the composition is independently and optionally an integer from one up to the number of amino acid residues to which conjugation is desired or targeted (e.g., the number of lysine residues) in the antibody (R²). In some embodiments, the value of n1 of each complex in the composition is independently and optionally selected from 1, 2, 3, 4, 5, 6, 7, 8, 9, 10, 11, 12, 13, 14, 15, 16, 17, 18, 19, 20, 21, 22, 23, 24, 25, 26, and 27. In some embodiments, the value of n1 of each complex in the composition is independently and optionally selected from 1, 2, 3, 4, 5, 6, 7, 8, 9, 10, 11, 12, 13, 14, 15, 16, 17, 18, 19, 20, 21, 22, 23, 24, 25 and 26. In some embodiments, the value of n1 of each complex in the composition is independently selected and optionally from an integer in the range of 1 to 27, 1 to 26, 1 to 10, 1 to 5, or 1 to 3. In some embodiments, the average value of n1 of complexes of the composition is in the range of 1 to 3, 1 to 5, 1 to 10, 1 to 26, or 1 to 27.

In some embodiments, a composition for administration to a subject in the methods described herein comprises unconjugated antibody (e.g., in trace amounts) and antibody conjugated to one or more oligonucleotides. In some embodiments, unconjugated antibody may be referred to as a compound of the structure of formula (I): [R¹]ₙ₁—R², for which n1 is zero. Accordingly, in some embodiments, a composition for administration to a subject in the methods described herein comprises compounds (e.g., complexes) of the structure of formula (I): [R¹]ₙ₁—R², for which each R¹ independently comprises a group comprising an oligonucleotide, R² comprises an anti-TfR1 antibody and n1 is independently an integer of zero or greater that reflects the number of instances of R¹ in each compound (e.g., complex). In some embodiments, the fraction of compounds of the structure of formula (I): [R¹]ₙ₁—R², in a composition, for which n1 is zero, compared with all compounds of that structure in the composition for which n1 is one or greater, is less than 10%, less than 5%, less than 1% less than 0.5%, less than 0.1%, less than 0.05%, or less than 0.01%.

In some embodiments, each instance of R¹ in a complex herein (e.g., a complex of a composition provided herein) is conjugated to a different amino acid residue of the antibody. In some embodiments, each different amino acid comprises an F-amino group (e.g., lysine, arginine). However, in some embodiments, each different amino acid to which R¹ is covalently linked is a cysteine. In some embodiments, each different amino acid to which R¹ is covalently linked is a lysine. In some embodiments, R¹ is directly covalently linked to an amino acid residue of the antibody. However, in some embodiments, R¹ is indirectly covalently linked to an amino acid of the antibody, e.g., covalently linked to a glycosylation site on the amino acid. In some embodiments, formulations are provided in which complexes for which R¹ is covalently linked to an amino acid residue residing in a CDR region of the antibody are present in only trace amounts, or in undetectable amount, or not at all. In some embodiments, formulations are provided in which complexes for which R¹ is covalently linked to an amino acid residue residing in a CDR region of the antibody are not detectable in the formulation using standard detection techniques.

In some embodiments, light chain constant regions of antibodies of complexes in a composition are independently covalently linked to an oligonucleotide at a linkage site represented by K188 (based on Kabat numbering) and/or a linkage site represented by K190 (based on Kabat numbering) of the light chain constant regions of the antibodies. In some embodiments, light chains of antibodies of complexes in a composition are independently covalently linked to an oligonucleotide at linkage sites represented by lysine (K) residues in a sequence motif DYEKHKVYA (SEQ ID NO: 27) of the light chains of the antibodies.

In some embodiments, compositions (e.g., in aqueous solutions) described herein comprise complexes that comprise a structure of the formula (I): [R¹]ₙ₁—R², wherein each R¹ in a complex of a composition provided herein independently comprises a group of the formula (Ia):

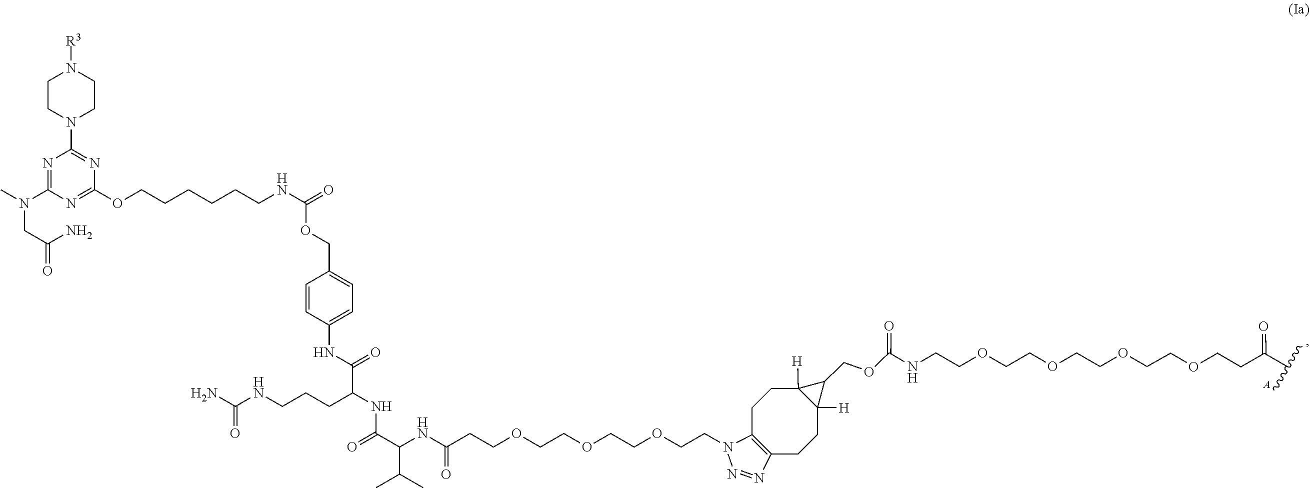

in which R³ is an oligonucleotide, e.g., a phosphorodiamidate morpholino oligomer (PMO); wherein in each complex n1 is independently an integer (e.g., of one or greater) representing the number of instances of R¹ in each complex, and each R¹ is covalently linked (e.g., indirectly or directly linked, e.g., directly linked) to R² at attachment point A. In some embodiments, R² comprises an anti-TfR1 antibody comprising a sequence as set forth in Table 2. For example, in some embodiments, R² comprises an anti-TfR1 antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody comprising a VH comprising the amino acid sequence of SEQ ID NO: 17 and/or comprising a VL comprising the amino acid sequence of SEQ ID NO: 18. In some embodiments, R² comprises an anti-TfR1 antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody comprising a heavy chain comprising the amino acid sequence of SEQ ID NO: 19 and/or comprising a light chain comprising the amino acid sequence of SEQ ID NO: 20. In some embodiments, R² comprises an anti-TfR1 antibody that is a Fab fragment, a full-length IgG, a Fab′ fragment, a F(ab′)₂ fragment, an scFv, or an Fv. In some embodiments, R² comprises an anti-TfR1 antibody that is a Fab fragment. In some embodiments, R³ is an oligonucleotide, e.g., a phosphorodiamidate morpholino oligomer (PMO) comprising the base sequence of CAATGCCATCCTGGAGTTCCTG (SEQ ID NO: 21). In some embodiments, each internucleoside linkage of the PMO is a phosphorodiamidate linkage. In some embodiments, R² comprises an anti-TfR1 antibody (e.g., a Fab) and each R¹ is covalently linked (e.g., indirectly or directly linked, e.g., directly linked) at attachment point A to a different amino acid residue of the antibody (e.g., Fab), optionally wherein each different amino acid residue is a lysine. In some embodiments, each R¹ is covalently linked to R² at attachment point A via a linkage site represented by a lysine (K) residue of the antibody. In some embodiments, a lysine (K) residue linkage site of the anti-TfR1 antibody is represented by K188 (based on Kabat numbering) and/or K190 (based on Kabat numbering) of a light chain constant region of the anti-TfR1 antibody. In some embodiments, a lysine (K) residue linkage site is represented by a lysine (K) residue in a sequence motif DYEKHKVYA (SEQ ID NO: 27) of a light chain of the anti-TfR1 antibody. In some embodiments, in each complex n1 is independently an integer (e.g., an integer in the range of 1-27, 1-26, 1-10, 1-5, or 1-3). In some embodiments, compositions (e.g., in aqueous solutions) for administration to a subject in the methods described herein comprise complexes that comprise a structure of formula (I): [R¹]ₙ₁—R², wherein n1 is 0.

In some embodiments, compositions (e.g., in aqueous solutions) described herein comprise complexes that comprise a structure of formula (I): [R¹]ₙ₁—R², in which each instance of R¹ in a complex of a composition provided herein comprises a group of the formula (Ib):

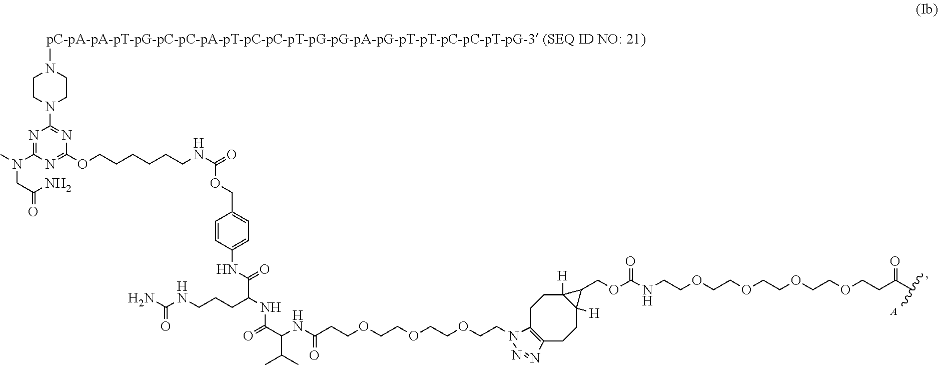

in which -p is a phosphorodiamidate linkage of a phosphorodiamidate morpholino oligomer (PMO), and wherein the PMO comprises a base sequence of CAATGCCATCCTGGAGTTCCTG (SEQ ID NO: 21); wherein in each complex n1 is independently an integer (e.g., of one or greater) representing the number of instances of R¹ in each complex, and each R¹ is covalently linked (e.g., indirectly or directly linked, e.g., directly linked) to R² at attachment point A. In some embodiments, R² comprises an anti-TfR1 antibody comprising a sequence as set forth in Table 2. For example, in some embodiments, R² comprises an anti-TfR1 antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody comprising a VH comprising the amino acid sequence of SEQ ID NO: 17 and/or comprising a VL comprising the amino acid sequence of SEQ ID NO: 18. In some embodiments, R² comprises an anti-TfR1 antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody comprising a heavy chain comprising the amino acid sequence of SEQ ID NO: 19 and/or comprising a light chain comprising the amino acid sequence of SEQ ID NO: 20. In some embodiments, R² comprises an anti-TfR1 antibody that is a Fab fragment, a full-length IgG, a Fab′ fragment, a F(ab′)₂ fragment, an scFv, or an Fv. In some embodiments, R² comprises an anti-TfR1 antibody that is a Fab fragment. In some embodiments, in each complex n1 is independently an integer (e.g., an integer in the range of 1-27, 1-26, 1-10, 1-5, or 1-3). In some embodiments, R² comprises an anti-TfR1 antibody (e.g., a Fab) and each R¹ is covalently linked (e.g., indirectly or directly linked, e.g., directly linked) at attachment point A to a different amino acid residue of the antibody (e.g., Fab), optionally wherein each different amino acid residue is a lysine. In some embodiments, each R¹ is covalently linked to R² at attachment point A via a linkage site represented by a lysine (K) residue of the antibody. In some embodiments, a lysine (K) residue linkage site of the anti-TfR1 antibody is represented by K188 (based on Kabat numbering) and/or K190 (based on Kabat numbering) of a light chain constant region of the anti-TfR1 antibody. In some embodiments, a lysine (K) residue linkage site is represented by a lysine (K) residue in a sequence motif DYEKHKVYA (SEQ ID NO: 27) of a light chain of the anti-TfR1 antibody. In some embodiments, compositions (e.g., in aqueous solutions) for administration to a subject in the methods described herein further comprise complexes that comprise a structure of formula (I): [R¹]ₙ₁—R², wherein n1 is 0.

In some embodiments, compositions (e.g., in aqueous solutions) described herein comprise complexes that comprise a structure of formula (I): [R¹]ₙ₁—R², in which each instance R¹ in a complex of a composition provided herein comprises a group of the formula (Ic):

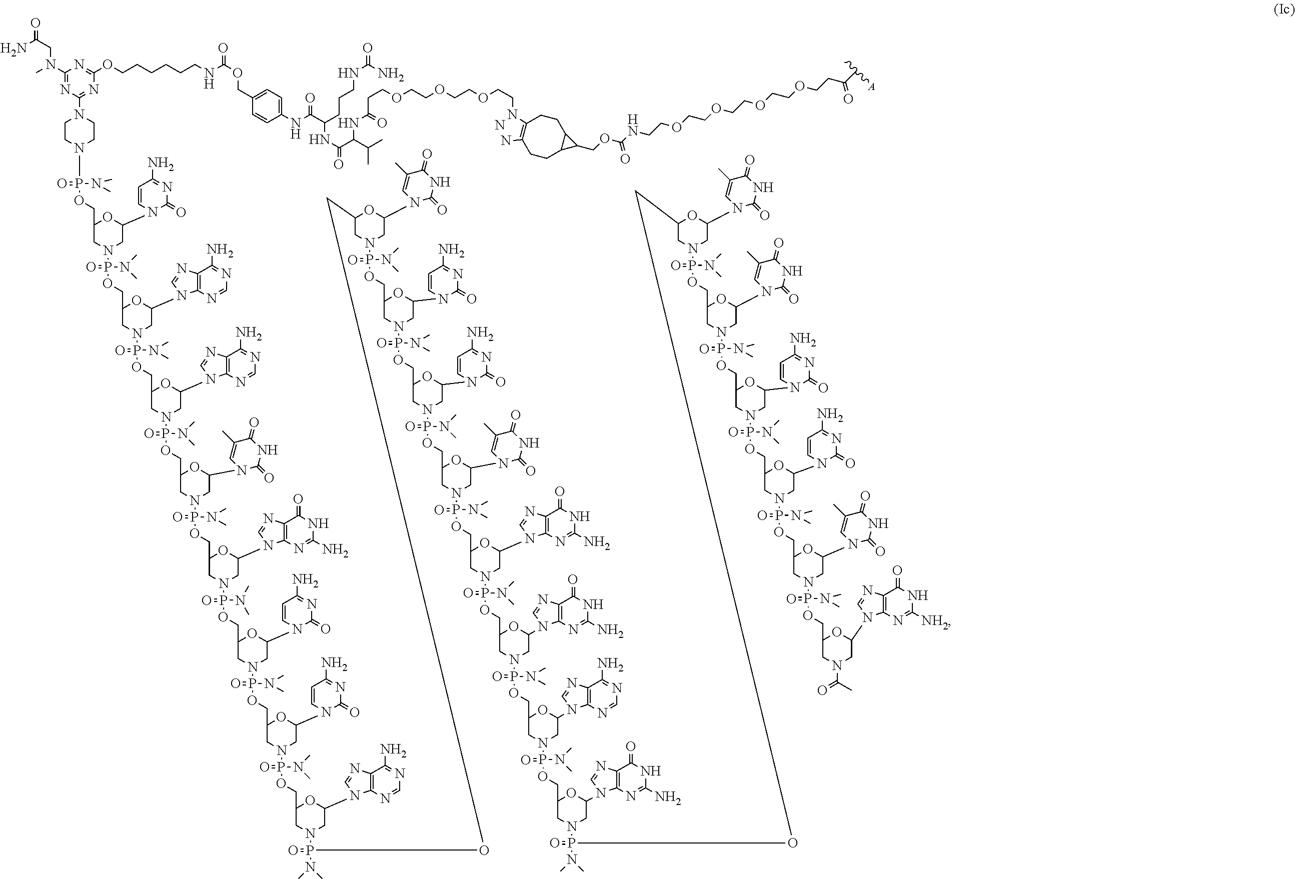

wherein in each complex n1 is independently an integer (e.g., of one or greater) representing the number of instances of R¹ in each complex, wherein each R¹ is covalently linked (e.g., indirectly or directly linked, e.g., directly linked) to R² at attachment point A. In some embodiments, R² comprises an anti-TfR1 antibody comprising a sequence as set forth in Table 2. For example, in some embodiments, R² comprises an anti-TfR1 antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody comprising a VH comprising the amino acid sequence of SEQ ID NO: 17 and/or comprising a VL comprising the amino acid sequence of SEQ ID NO: 18. In some embodiments, R² comprises an anti-TfR1 antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody comprising a heavy chain comprising the amino acid sequence of SEQ ID NO: 19 and/or comprising a light chain comprising the amino acid sequence of SEQ ID NO: 20. In some embodiments, R² comprises an anti-TfR1 antibody that is a Fab fragment, a full-length IgG, a Fab′ fragment, a F(ab′)₂ fragment, an scFv, or an Fv. In some embodiments, R² comprises an anti-TfR1 antibody that is a Fab fragment. In some embodiments, in each complex n1 is independently an integer (e.g., an integer in the range of 1-27, 1-26, 1-10, 1-5, or 1-3). In some embodiments, R² comprises an anti-TfR1 antibody (e.g., a Fab) and each R¹ is covalently linked (e.g., indirectly or directly linked, e.g., directly linked) at attachment point A to a different amino acid residue of the antibody (e.g., Fab), optionally wherein each different amino acid residue is a lysine. In some embodiments, each R¹ is covalently linked to R² at attachment point A via a linkage site represented by a lysine (K) residue of the antibody. In some embodiments, a lysine (K) residue linkage site of the anti-TfR1 antibody is represented by K188 (based on Kabat numbering) and/or K190 (based on Kabat numbering) of a light chain constant region of the anti-TfR1 antibody. In some embodiments, a lysine (K) residue linkage site is represented by a lysine (K) residue in a sequence motif DYEKHKVYA (SEQ ID NO: 27) of a light chain of the anti-TfR1 antibody. In some embodiments, compositions (e.g., in aqueous solutions) for administration to a subject in the methods described herein further comprise complexes that comprise a structure of formula (I): [R¹]ₙ₁—R², wherein n1 is 0.

In some embodiments, compositions (e.g., in aqueous solutions) described herein comprise complexes that comprise a structure of formula (Id):

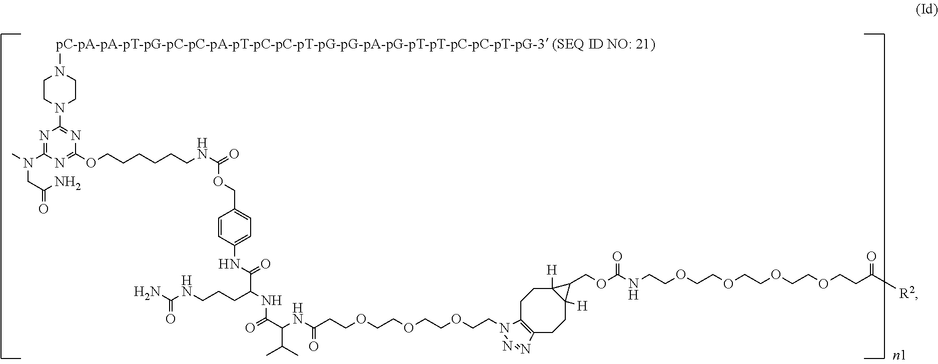

in which -p is a phosphorodiamidate linkage of a phosphorodiamidate morpholino oligomer (PMO), and wherein the PMO comprises a base sequence of CAATGCCATCCTGGAGTTCCTG (SEQ ID NO: 21); wherein R² comprises an anti-TfR11 antibody (e.g., a Fab) comprising a CDR-H1, a CDR-H2, a CDR-H3, a CDR-L1, a CDR-L2, and a CDR-L3 selected from Table 2, optionally wherein the antibody (e.g., a Fab) comprises a VH comprising the amino acid sequence of SEQ ID NO: 17 and a VL comprising the amino acid sequence of SEQ ID NO: 18, further optionally wherein the antibody (e.g., a Fab) comprises a heavy chain comprising the amino acid sequence of SEQ ID NO: 19 and a light chain comprising the amino acid sequence of SEQ ID NO: 20; and wherein in each complex n1 is independently an integer (e.g., of one or greater) representing the number of instances of the group enclosed by square brackets, wherein each instance of the group enclosed by square brackets is covalently linked to a different amino acid residue of the antibody (e.g., a Fab), optionally wherein each different amino acid residue is a lysine. In some embodiments, R² comprises an anti-TfR1 antibody (e.g., a Fab) comprising a sequence as set forth in Table 2. For example, in some embodiments, R² comprises an anti-TfR1 antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an anti-TfR1 antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an anti-TfR1 antibody (e.g., a Fab) comprising a VH comprising the amino acid sequence of SEQ ID NO: 17 and/or comprising a VL comprising the amino acid sequence of SEQ ID NO: 18. In some embodiments, R² comprises an anti-TfR1 antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an anti-TfR1 antibody (e.g., a Fab) comprising a heavy chain comprising the amino acid sequence of SEQ ID NO: 19 and/or comprising a light chain comprising the amino acid sequence of SEQ ID NO: 20. In some embodiments, in each complex n1 is independently an integer (e.g., an integer in the range of 1-27, 1-26, 1-10, 1-5, or 1-3). In some embodiments, in each complex n1 is independently an integer of one or greater. In some embodiments, R² comprises an anti-TfR1 antibody (e.g., a Fab) that is covalently linked (e.g., indirectly or directly linked, e.g., directly linked) via different amino acid residue of the antibody (e.g., Fab), optionally wherein each different amino acid residue is a lysine. In some embodiments, each instance of the group enclosed by square brackets in the structure of formula (Id) is covalently linked to a linkage site represented by a lysine (K) residue of the anti-TfR1 antibody (e.g., the Fab). In some embodiments, a lysine (K) residue linkage site of the anti-TfR1 antibody is represented by K188 (based on Kabat numbering) and/or K190 (based on Kabat numbering) of a light chain constant region of the anti-TfR1 antibody. In some embodiments, a lysine (K) residue linkage site is represented by a lysine (K) residue in a sequence motif DYEKHKVYA (SEQ ID NO: 27) of a light chain of the anti-TfR1 antibody. In some embodiments, compositions (e.g., in aqueous solutions) for administration to a subject in the methods described herein further comprise complexes in which n1 is 0.

In some embodiments, compositions (e.g., aqueous solutions) described herein comprise a structure of formula (A):

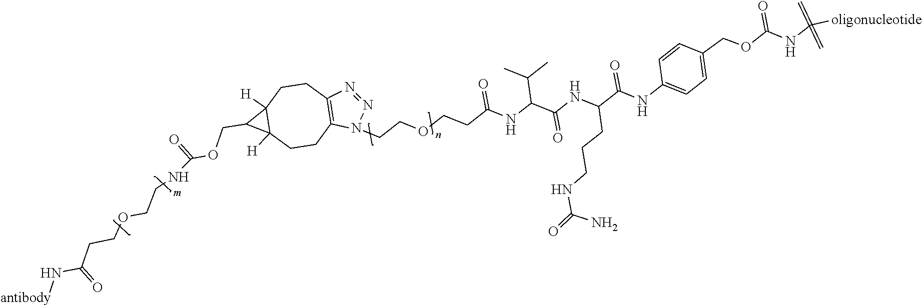

(A), wherein n is 0-15 (e.g., 3) and m is 0-15 (e.g., 4). In some embodiments, the antibody is an an-TfR1 antibody (e.g., the anti-TfR1 antibody provided in Table 2). In some embodiments, the oligonucleotide is a PMO and comprises the base sequence of SEQ ID NO: 21. In some embodiments, each internucleoside linkage of the PMO is a phosphorodiamidate linkage. In some embodiments, the amide shown adjacent to the antibody in the structure results from a reaction with an amine of the antibody, such as a lysine epsilon amine. In some embodiments, the antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the antibody comprises a VH comprising the amino acid sequence of SEQ ID NO: 17 and/or comprises a VL comprising the amino acid sequence of SEQ ID NO: 18. In some embodiments, the antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)₂ fragment, an scFv, or an Fv.

In each composition disclosed herein, complexes (e.g., complexes comprising a structure of formula (I): [R¹]ₙ₁—R², such as complexes in which each R^{t} comprises a group of the formula (Ia), (Ib), or (Ic); complexes comprising a structure of formula (Id); or complexes comprising a structure of formula (A)) of the composition may comprise a structure having the stereochemistry shown in formula (B):

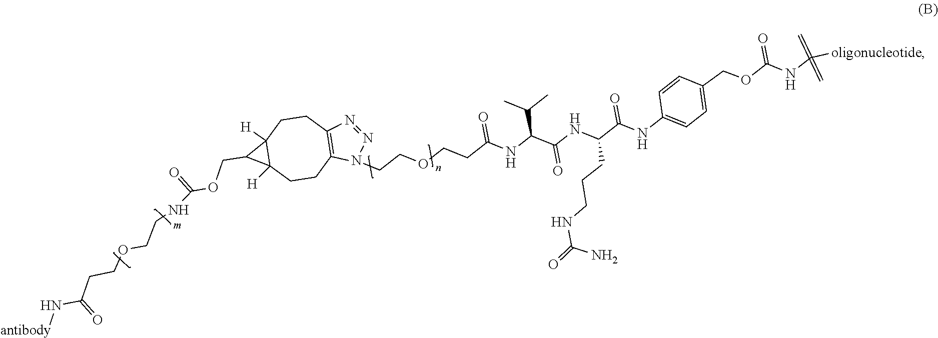

wherein n is 0-15 (e.g., 3) and m is 0-15 (e.g., 4). It should be understood that the stereochemistry shown in formula (B) can be applied to the corresponding portion of any formula or structure provided herein (e.g., formula (Ia), (Ib), (Ic), (Id), or (A)).

In some embodiments, light chain constant regions of antibodies of complexes (e.g., complexes comprising a structure of formula (I): [R¹]ₙ₁—R², such as complexes in which each R^{t} comprises a group of the formula (Ia), (Ib), or (Ic); complexes comprising a structure of formula (Id); or complexes comprising a structure of formula (A)) in a composition disclosed herein are independently covalently linked to an oligonucleotide at linkage sites represented by K188 (based on Kabat numbering) and/or K190 (based on Kabat numbering) of the light chain constant regions of the antibodies of the complexes. In some embodiments, light chains of antibodies of complexes (e.g., complexes comprising a structure of formula (I): [R¹]ₙ₁—R², such as complexes in which each R^{t} comprises a group of the formula (Ia), (Ib), or (Ic); complexes comprising a structure of formula (Id); or complexes comprising a structure of formula (A)) in a composition disclosed herein are independently covalently linked to oligonucleotides at linkage sites represented by lysine (K) residues in a sequence motif DYEKHKVYA (SEQ ID NO: 27) of the light chains of the antibodies of the complexes.

In some embodiments, provided is a product (e.g., lyophilized composition described herein), produced by a process comprising lyophilizing an aqueous solution of a composition (e.g., in aqueous form) described herein.

In some embodiments, a composition is formulated to be compatible with its intended route of administration. Examples of routes of administration include parenteral, e.g., intravenous, intradermal, subcutaneous, administration. Typically, the route of administration is intravenous or subcutaneous.

### Methods of Use/Treatment

Complexes comprising an anti-TfR1 antibody (e.g., Fab) covalently linked to an oligonucleotide (e.g., a phosphorodiamidate morpholino oligomer (PMO)) as described herein are effective in treating a subject having a dystrophinopathy, e.g., Duchenne Muscular Dystrophy. In some embodiments, complexes comprise an oligonucleotide that facilitates exon skipping of an mRNA expressed from a mutated DMD allele.

In some embodiments, a subject may be a human subject, a non-human primate subject, a rodent subject, or any suitable mammalian subject. In some embodiments, a subject may have Duchenne muscular dystrophy or other dystrophinopathy. In some embodiments, a subject has a mutated DMD allele, which may optionally comprise at least one mutation in a DMD exon that causes a frameshift mutation and leads to improper RNA splicing/processing. In some embodiments, a subject is suffering from symptoms of a severe dystrophinopathy, e.g. muscle atrophy or muscle loss. In some embodiments, a subject has an asymptomatic increase in serum concentration of creatine phosphokinase (CK) and/or (e.g., and) muscle cramps with myoglobinuria. In some embodiments, a subject has a progressive muscle disease, such as Duchenne or Becker muscular dystrophy or DMD-associated dilated cardiomyopathy (DCM). In some embodiments, a subject is not suffering from symptoms of a dystrophinopathy.

In some embodiments, a subject has a mutation in a DMD gene that is amenable to exon 45 skipping. In some embodiments, a complex as described herein is effective in treating a subject having a mutation in a DMD gene that is amenable to exon 45 skipping. In some embodiments, a complex comprises an oligonucleotide, e.g., an antisense oligonucleotide that facilitates skipping of exon 45 of a pre-mRNA, such as in a pre-mRNA encoded from a mutated DMD gene (e.g., a mutated DMD gene that is amenable to exon 45 skipping).

In some embodiments, a pharmaceutical composition comprising a complex as described herein may be administered by a suitable route, which may include intravenous administration, e.g., as a bolus or by continuous infusion over a period of time. In some embodiments, intravenous administration may be performed by intramuscular, intraperitoneal, intracerebrospinal, subcutaneous, intra-articular, intrasynovial, or intrathecal routes. In some embodiments, a pharmaceutical composition may be in solid form, aqueous form, or a liquid form. In some embodiments, an aqueous or liquid form may be nebulized or lyophilized. In some embodiments, a nebulized or lyophilized form may be reconstituted with an aqueous or liquid solution.

Compositions for intravenous administration may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the complex and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the complex, can be dissolved and administered in a pharmaceutical excipient such as Water-for-Injection, 0.9% saline, or 5% glucose solution.

In some embodiments, a pharmaceutical composition that comprises a complex comprising a muscle-targeting agent (e.g., an anti-TfR1 antibody, e.g., an anti-TfR1 Fab) covalently linked to an oligonucleotide (e.g., a PMO) is administered via site-specific or local delivery techniques. Examples of these techniques include implantable depot sources of the complex, local delivery catheters, site specific carriers, direct injection, or direct application.

In some embodiments, a pharmaceutical composition that comprises a complex comprising a muscle-targeting agent (e.g., an anti-TfR1 antibody, e.g., an anti-TfR1 Fab) covalently linked to an oligonucleotide (e.g., a PMO) is administered at an effective concentration that confers therapeutic effect on a subject. Effective amounts vary, as recognized by those skilled in the art, depending on the severity of the disease, unique characteristics of the subject being treated, e.g. age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g. the half-life of the complex in a subject, generally will contribute to determination of the concentration of pharmaceutical composition that is used for treatment. The frequency of administration may be empirically determined and adjusted to maximize the efficacy of the treatment.

The efficacy of treatment may be assessed using any suitable methods. In some embodiments, the efficacy of treatment may be assessed by evaluation of observation of symptoms associated with a dystrophinopathy, e.g., muscle atrophy or muscle weakness, through measures of a subject's self-reported outcomes, e.g., mobility, self-care, usual activities, pain/discomfort, and anxiety/depression, or by quality-of-life indicators, e.g., lifespan.

In some embodiments, a pharmaceutical composition that comprises a complex comprising a muscle-targeting agent (e.g., an anti-TfR1 antibody, e.g., an anti-TfR1 Fab) covalently linked to an oligonucleotide (e.g., a PMO) described herein is administered to a subject at an effective concentration sufficient to modulate activity or expression of a target gene by at least 10%, at least 20%, at least 30%, at least 40%, at least 50%, at least 60%, at least 70%, at least 80%, at least 90% or at least 95% relative to a control, e.g. baseline level of gene expression prior to treatment.

### EXAMPLES

### Example 1. Delivery of DMD-Targeting Oligonucleotide to Muscle Tissue Following Systemic Administration to Non-Human Primates

This study evaluated the delivery of conjugates (referred to in this Example as “anti-TfR1 Fab-ASO conjugate(s)”) comprising the anti-TfR1 Fab having the heavy chain and light chain sequences shown in Table 2 covalently linked (through lysine conjugation) via a cleavable linker comprising a Valine-Citrulline sequence to a DMD exon 45-skipping oligonucleotide (the oligonucleotide is referred to as “ASO” in this Example). The ASO is a PMO and comprises the base sequence of SEQ ID NO: 21. The anti-TfR1 Fab-ASO conjugates comprise a structure of:

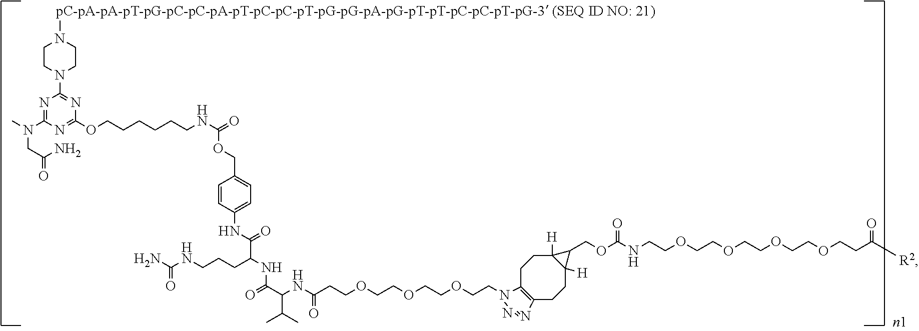

in which -p is a phosphorodiamidate linkage of the ASO, and wherein R² comprises the anti-TfR1 Fab.

ASO distribution in muscle tissue was tested in vivo in healthy non-human primates. Naïve male cynomolgus monkeys (n=4 per group) were administered two doses of 30 mg/kg of ASO not covalently linked to an antibody (“naked”), or anti-TfR1 Fab-ASO conjugates at a dose equivalent to 30 mg/kg ASO, via intravenous infusion on weeks 0 and 4. Animals were sacrificed, and tissues harvested 4 weeks after the second dose was administered (i.e., 8 weeks after the first dose).

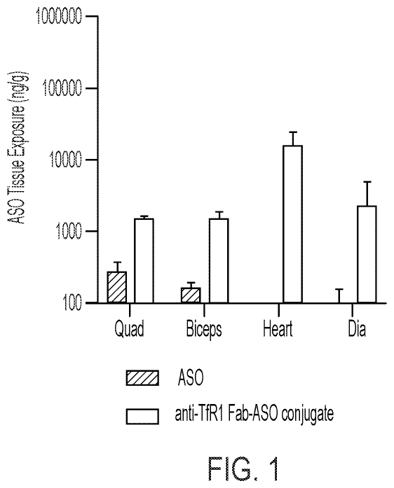

Tissue ASO accumulation was quantified using a hybridization ELISA with a probe complementary to the ASO sequence. A standard curve was generated and ASO levels (in ng/g) were derived from a linear regression of the standard curve. The ASO was distributed to all tissues evaluated at a higher level following the administration of the anti-TfR1 Fab-ASO conjugates as compared to the administration of naked ASO. Intravenous administration of naked ASO resulted in levels of ASO at the timepoint evaluated that were close to background levels in heart and diaphragm (“Dia”) and were very low in quadriceps (“Quad”) and biceps. Administration of anti-TfR1 Fab-ASO conjugates resulted in substantial accumulation of ASO in the measured tissues at the timepoint evaluated, with a rank order of heart>diaphragm (“Dia”)>quadriceps (“Quad”)>biceps (FIG. 1).

The results demonstrate that a complex comprising an anti-TfR1 antibody (e.g., a Fab) covalently linked to an oligonucleotide (e.g., a PMO) achieve enhanced delivery to muscle tissue of the oligonucleotide following systemic administration, when compared to administration of ASO not comprised within a complex (i.e., not covalently linked to an anti-TfR1 antibody).

### Example 2. Exon 45 Skipping in Human Skeletal Muscle Myoblasts

This study evaluated skipping of exon 45 in the DMD transcript following treatment with conjugates (referred to in this Example as “anti-TfR1 Fab-ASO conjugates”) as described in Example 1 (comprising an anti-TfR1 Fab covalently linked to a DMD exon 45-skipping oligonucleotide (“ASO”)).

Human skeletal muscle myoblasts (obtained from Association Institut de Myologie) were modified using CRISPR/Cas to introduce a deletion of exon 46 in the DMD gene to reflect common mutations in Duchenne Muscular Dystrophy patients amenable to exon 45 skipping, generating a mutated myoblast cell line (“Del46”). The wild-type and mutated myoblasts were used to generate myotubes (“WT Myotubes” and “Del46 Myotubes” respectively).

WT and Del46 Myotubes were treated with anti-TfR1 Fab-ASO conjugates at an ASO-equivalent concentration of 10 M. Following incubation with the conjugates, total RNA was harvested and cDNA synthesis was performed. End-point PCR was conducted to evaluate the degree of exon 45 skipping in the cells. PCR products were quantified using capillary electrophoresis and the relative amounts of the skipped and unskipped amplicon were used to generate a ratio of the Exon 45-skipped amplicon relative to the total amount of amplicon present:

$$\% Exon Skipping = \frac{Skipped Amplicon}{( Skipped Amplicon + Unskipped Amplicon )} * 100 .$$

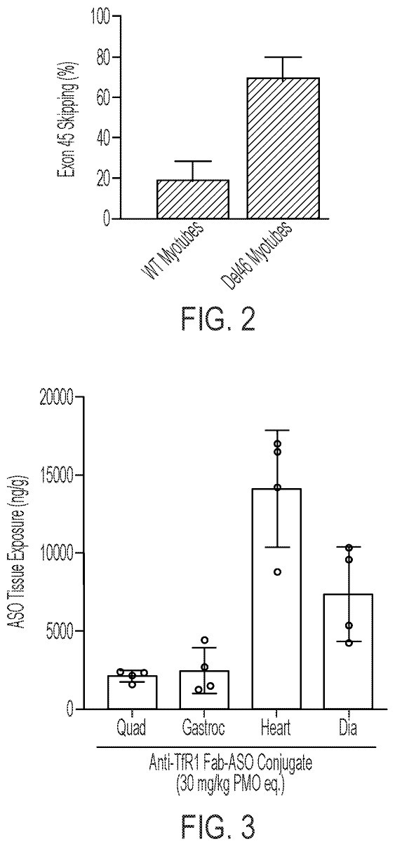

The results demonstrate that the anti-TfR1 Fab-ASO conjugates induce greater exon 45 skipping in myotubes comprising DMD mutations amenable to exon 45 skipping than in wild-type myotubes (FIG. 2). Measured exon 45 skipping in wild-type myotubes was approximately 18% following treatment with anti-TfR1 Fab-ASO conjugates, compared with approximately 70% in Del46 myotubes. This indicates that the anti-TfR1 Fab-ASO conjugates enabled exon 45 skipping in the myotubes, and that such skipping was more pronounced in myotubes comprising DMD mutations amenable to exon 45 skipping.

### Example 3. Delivery of DMD-Targeting Oligonucleotide to Muscle Tissue Following Systemic Administration to Humanized Mouse Model of Duchenne Muscular Dystrophy and Resultant Exon 45 Skipping

This study evaluated delivery to muscle tissue and subsequent exon 45 skipping following a single intravenous administration of conjugates (referred to in this Example as “anti-TfR1 Fab-ASO conjugates”) as described in Example 1 (comprising anti-TfR1 Fab covalently linked to DMD exon 45-skipping oligonucleotide (“ASO”)).

hDMD_{WT}/mdx mice (obtained from Academisch Ziekenhuis Leiden) were crossed with mice expressing human TfR1, to generate hTfR1/hDMD_{WT}/mdx mice. The hTfR1/hDMD_{WT}/mdx mice were administered anti-TfR1 Fab-ASO conjugates intravenously at an ASO-equivalent dose of 30 mg/kg. Concentrations of the ASO and exon 45 skipping were measured in various muscle tissues, 7 days following a single dose of the conjugates. Tissue exposure (in ng ASO per g of tissue) of the ASO was measured by hybridization ELISA (Burki et al., Nucleic Acid Ther. 2015 October; 25(5):275-84, incorporated herein by reference). Exon 45 skipping was measured using end-point PCR as explained in Example 2 above.

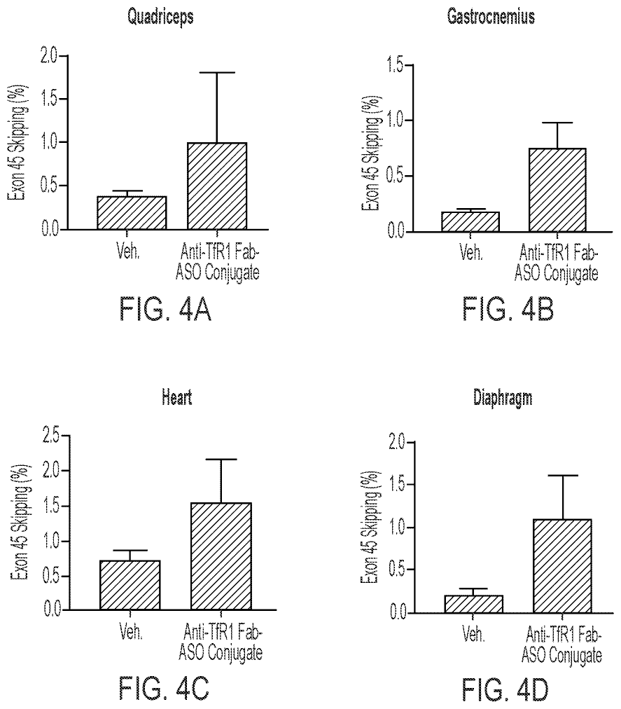

The results demonstrate that ASO accumulated within various muscle tissues following a single dose of the conjugates. Tissue exposure measured in the muscle tissues were approximately 2,000 ng/g in quadriceps (“Quad”), 2,400 ng/g in gastrocnemius (“Gastroc”), 14,000 ng/g in heart (“Heart”), and 7,300 ng/g in diaphragm (“Dia”) (FIG. 3). Furthermore, enhanced exon 45 skipping was observed in each muscle tissue tested in mice treated with the conjugates compared to those treated with vehicle control (“Veh.”). Exon 45 skipping in vehicle-versus conjugate-treated mice was approximately 0.4% vs. 1.0% in quadriceps (FIG. 4A); 0.2% vs. 0.75% in gastrocnemius (FIG. 4B); 0.7% vs. 1.5% in heart (FIG. 4C); and 0.2% vs. 1.2% in diaphragm (FIG. 4D).

These results indicate that conjugates comprising an anti-TfR1 antibody (e.g., a Fab) covalently linked to an oligonucleotide (e.g., a PMO, such as an exon-skipping PMO) achieve delivery to muscle tissue of the oligonucleotide following systemic administration, and that such conjugates comprising an exon-skipping oligonucleotide (e.g., an exon 45-skipping PMO oligonucleotide) induce exon skipping in muscle tissues following systemic delivery.

### EQUIVALENTS AND TERMINOLOGY

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or (e.g., and) one or more modified nucleotides and/or (e.g., and) one or more modified internucleoside linkages and/or (e.g., and) one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

What is claimed is:

1. A complex comprising a structure of formula (I): [R¹]ₙ₁—R², wherein each R¹ comprises a group of the formula (Ia): 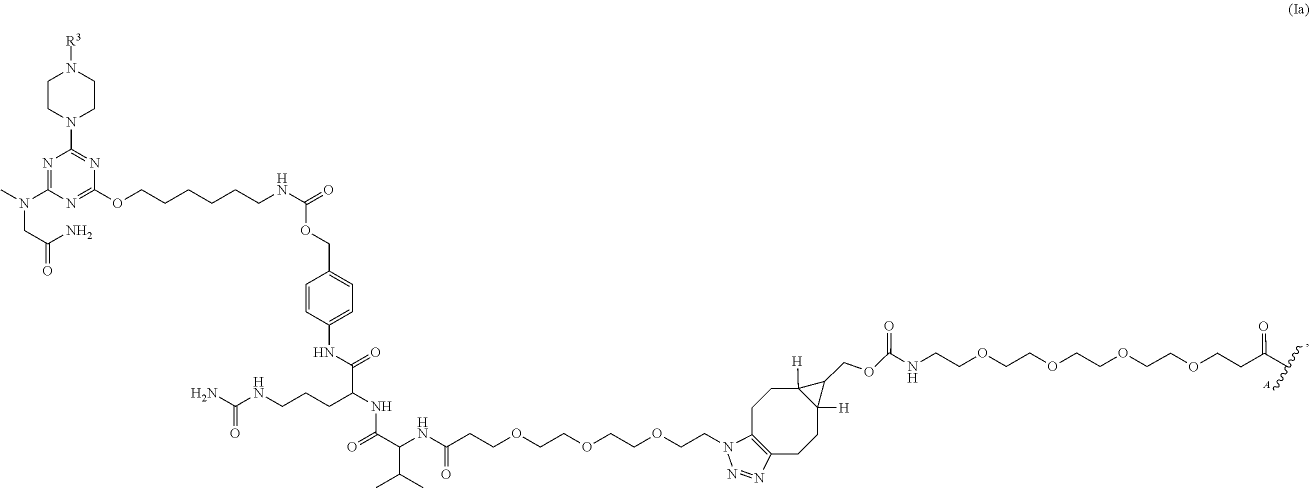
wherein R³ comprises a phosphorodiamidate morpholino oligomer (PMO) comprising the base sequence of CAATGCCATCCTGGAGTTCCTG (SEQ ID NO: 21);
wherein R² comprises an anti-transferrin receptor 1 (anti-TfR1) antibody comprising a heavy chain complementarity determining region 1 (CDR-H1), a heavy chain complementarity determining region 2 (CDR-H2), a heavy chain complementarity determining region 3 (CDR-H3), a light chain complementarity determining region 1 (CDR-L1), a light chain complementarity determining region 2 (CDR-L2), and a light chain complementarity determining region (CDR-L3) selected from Table 2,
wherein R¹ is covalently linked to R² at attachment point A; and wherein n1 is an integer of one or greater representing the number of instances of R¹ in the complex, wherein each instance of R¹ is covalently linked to a different amino acid residue of the anti-TfR1 antibody.

2. A complex comprising a structure of formula (I): [R¹]ₙ₁—R², wherein each R¹ comprises a group of the formula (Ib): 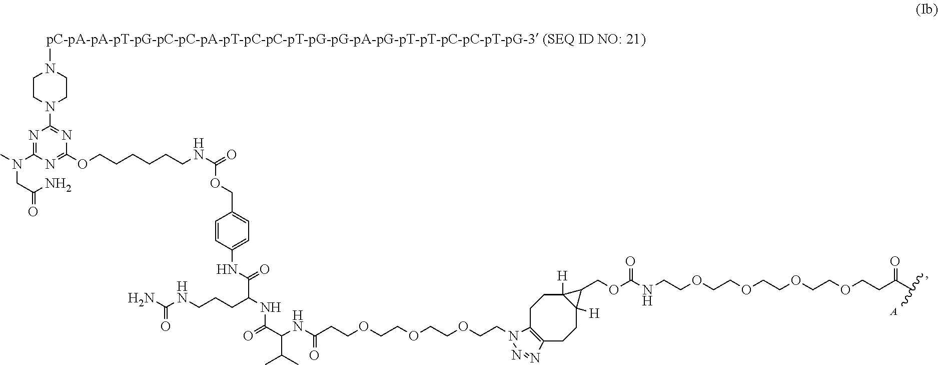
in which -p is a phosphorodiamidate linkage of a phosphorodiamidate morpholino oligomer (PMO), and wherein the PMO comprises a base sequence of
(SEQ ID NO: 21)
CAATGCCATCCTGGAGTTCCTG;
wherein R² comprises an anti-TfR1 antibody comprising a CDR-H1, a CDR-H2, a CDR-H3, a CDR-L1, a CDR-L2, and a CDR-L3 selected from Table 2;
wherein R¹ is covalently linked to R² at attachment point A; and wherein n1 is an integer of one or greater representing the number of instances of R¹, wherein each instance of R¹ is covalently linked to a different amino acid residue of the anti-TfR1 antibody.

3. A complex comprising a structure of formula (I): [R¹]ₙ₁—R², wherein each R¹ comprises a group of the formula (Ic): 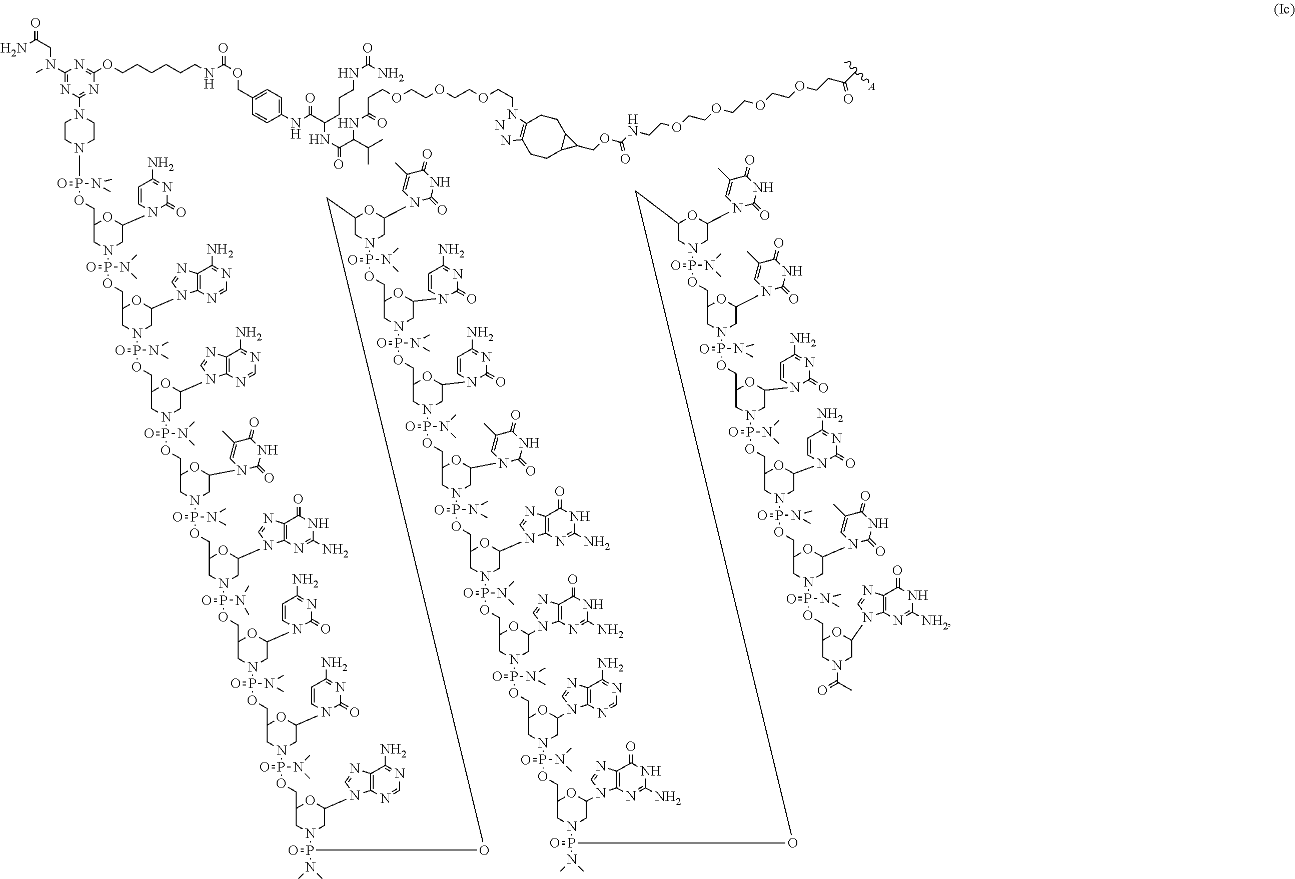
wherein R² comprises an anti-TfR1 antibody comprising a CDR-H1, a CDR-H2, a CDR-H3, a CDR-L1, a CDR-L2, and a CDR-L3 selected from Table 2;
wherein R¹ is covalently linked to R² at attachment point A; and wherein n1 is an integer of one or greater representing the number of instances of R¹, wherein each instance of R¹ is covalently linked to a different amino acid residue of the anti-TfR1 antibody.

4. A complex comprising a structure of formula (Id). 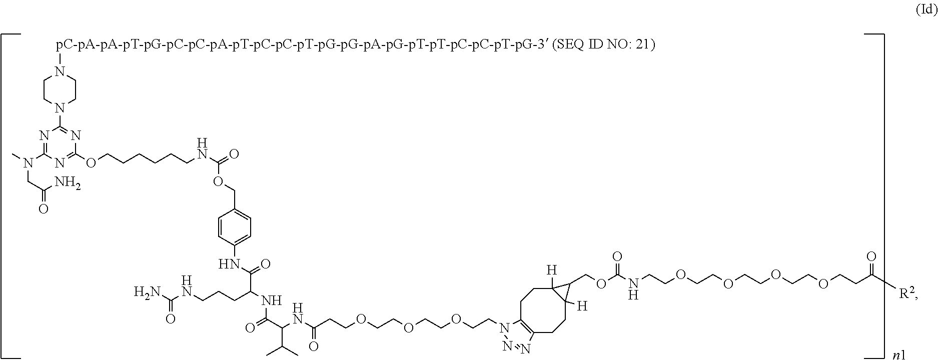
in which -p is a phosphorodiamidate linkage of a phosphorodiamidate morpholino oligomer (PMO), and wherein the PMO comprises a base sequence of
(SEQ ID NO: 21)
CAATGCCATCCTGGAGTTCCTG;
wherein R² comprises an anti-TfR1 antibody comprising a CDR-H1, a CDR-H2, a CDR-H3, a CDR-L1, a CDR-L2, and a CDR-L3 selected from Table 2,
wherein each instance of the group enclosed by square brackets in formula (Id) is covalently linked to a different amino acid residue of the anti-TfR1 antibody; and wherein n1 is an integer of one or greater representing the number of instances of the group enclosed by square brackets in formula (Id).

5. The complex of any one of claims 1 to 4, wherein the anti-TfR1 antibody is a Fab fragment, a full-length IgG, a Fab′ fragment, or a F(ab′)₂ fragment.

6. The composition of any one of claims 1 to 5, wherein the anti-TfR1 antibody is a Fab fragment.

7. The complex of any one of claims 1 to 6, wherein the anti-TfR1 antibody comprises a VH comprising the amino acid sequence of SEQ ID NO: 17 and a VL comprising the amino acid sequence of SEQ ID NO: 18.

8. The complex of any one of claims 1 to 7, wherein the anti-TfR1 antibody comprises a heavy chain comprising the amino acid sequence of SEQ ID NO: 19 and a light chain comprising the amino acid sequence of SEQ ID NO: 20.

9. The complex of any one of claims 1 to 8, wherein each instance of R¹ is covalently linked to a different lysine residue of the anti-TfR1 antibody.

10. The complex of any one of claims 1 to 9, wherein the different amino acid residues comprise K188 and K190 of the light chain constant region based on Kabat numbering.

11. The complex of any one of claims 1 to 10, wherein the different amino acid residues are represented by lysine (K) residues in a sequence motif DYEKHKVYA (SEQ ID NO: 27) of the light chain constant region of the anti-TfR1 antibody.

12. A composition comprising the complex of any one of claims 1 to 11, optionally wherein the composition is in the form of an aqueous solution.

13. The composition of claim 12, wherein the anti-TfR1 antibodies of the complexes in the composition comprise light chain constant regions, and wherein at least 80% of the light chain constant regions of the anti-TfR1 antibodies of the complexes in the composition are independently covalently linked to an oligonucleotide at a linkage site represented by K188 (based on Kabat numbering) and/or a linkage site represented by K190 (based on Kabat numbering) of the light chain constant region of each anti-TfR1 antibody.

14. The composition of claim 12, wherein the anti-TfR1 antibodies of the complexes in the composition comprise light chain constant regions, and wherein at least 80% of light chain constant regions of the anti-TfR1 antibodies of the complexes in the composition are independently covalently linked to an oligonucleotide at linkage sites represented by lysine (K) residues in a sequence motif DYEKHKVYA (SEQ ID NO: 27) of the light chain constant region of each antibody.

15. A method of promoting expression or activity of a dystrophin protein in a subject, the method comprising administering to the subject the complex of any one of claims 1 to 11 or the composition of any one of claims 12 to 14.

16. The method of claim 15, wherein the dystrophin protein is a truncated dystrophin protein.

17. A method of treating a subject having a mutated DMD allele associated with Duchenne Muscular Dystrophy, the method comprising administering to the subject the complex of any one of claims 1 to 11 or the composition of any one of claims 12 to 14.

18. The method of claim 17, wherein the complex promotes expression or activity of a dystrophin protein in the subject.

19. The method of claim 18, wherein the dystrophin protein is a truncated dystrophin protein.

20. The method of any one of claims 17 to 19, wherein the mutated DMD allele comprises a mutation amenable to exon 45 skipping.

21. The method of any one of claims 17 to 20, wherein the mutated DMD allele comprises a frameshift mutation in exon 45.
